SEQUENCE LISTING (17 sequences)

SEQ ID NO: 1 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 2 - protein, 394 aa, Ralstonia eutropha
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 3 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 4 - protein, 401 aa, Thermus thermophilus
MPEAWIVEAVRTPIGKHGGALASVRPDDLLAHALSVLVDRSGVPKEEVEDVYAGCANQAGEDNRNVARMALLLAGFPVEVAGCTVNRLCGSGLEAVAQAARAIWAGEGKVYIGSGVESMSRAPYAVPKPERGFPTGNLVMYDTTLGWRFVNPKMQALYGTESMGETAENLAEMYGIRREEQDRFALLSHQKAVRAWEEGRFQDEVVPVPVKRGKEEILVEQDEGPRRDTSLEKLAALRPVFREGGTVTAGNSSPLNDGAAAVLLVSDDYAKAHGLRPLARVRAIAVAGVPPRIMGIGPVPATRKALERAGLSFSDLGLIELNEAFAAQALAVLREWSLSMEDQRLNPNGGAIALGHPLGASGARILTTLVHEMRRRKVQFGLATMCIGVGQGIAVVVEGMG

SEQ ID NO: 5 - protein, 402 aa, Flavobacteriaceae bacterium
MPYQKTVKYIMQEVFIVSAVRTPIGSFLGSLAGVPATKLGSAAIKGALEKINLDPNQVQEVYMGNVLQAGEGQAPARQAALGAGLSNTTPATTVNKVCASGMKAVMMASQAIKCGDQDVVVAGGMENMSLVPHYFNARSSTKLGEVKMQDGMILDGLTDVYGKVHMGVCAEKCAAENGFSREDQDNFAVESYKRSAKAWADGKFKDEVVAVEIPQRKGDPVIFAEDEEYKSVNFDRISTLPTVFQKENGTVTAANASTINDGASALILMSKDKMEELGLKPLAKIVSYADAAHEPEWFTTAPSKALPIALKKAGLEVSDIDYFEFNEAFSVVGLANNKILGLDASKVNVNGGAVSLGHPLGSSGSRIIVTLINILKQNNGKYGAAAICNGGGGASAIVIENV

SEQ ID NO: 6 - protein, 255 aa, Pseudomonas putida
MHIANKHFIVSGAASGLGAATAQMLVEAGAKVMLVDLNAQAVEAKARELGDNARFAVADISDEQAAQSAVDAAVSAFGSLHGLVNCAGIVGAEKVLGKQGPHGLASFAKVINVNLIGSFNLLRLAAAAMAEGAADESGERGVIINTASIAAYDGQIGQAAYAASKGAIASLTLPAARELARFGIRVMTIAPGIFETPMMAGMSEEVRASLAAGVPFPPRLGRPQEYAALARHIIENSMLNGEVIRLDGALRMAAK

SEQ ID NO: 7 - protein, 394 aa, Ralstonia eutropha
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAAGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 8 - protein, 394 aa, Ralstonia eutropha
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKALGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 9 - protein, 364 aa, Ascaris suum
MATSRLVCSNLTKQCFTISSRAASQFTVFFGCVVPSNCGQGPARQATLGAGCDPSTIVTTLNKLCASGMKSIACAASLLQLGLQEVTVGGGMESMSLVPYYLERGETTYGGMKLIDGIPRDGLTDAYSNQLMGACADNVAKRFNITREEQDKFAIESYKRSAAAWESGACKAEVVPIEVTKGKKTYIVDKDEEYTKVNFEKLPKLKPAFLKDGTITAGNASTLNDGAAAVVMTTVEGAKKYGVKPLARLLSYGDAATNPVDFAIAPSMVIPKVLKLANLEIKDIDLWEINEAFAVVPLHSMKTLGIDHSKVNIHGGGVSLGHPIGMSGARIIVHLIHALKPGQKGCAAICNGGGGAGGMVIEKL

SEQ ID NO: 10 - protein, 392 aa, Chryseobacterium aquaticum
MKEVFIVSAVRTPMGSFMGSLSTVPATKLGATAVKGALDKISLDPKNVQEIYMGNVLQAGEGQAPARQVALGAGLSNETPSTTINKVCASGMKAVTMAAQAIKAGDADVIVAGGMENMSMVPHYYNARNATKLGDVKMQDGMVLDGLTDVYNKVHMGVCAEKCATDYSFSREDQDNFAIESYKRSAKAWSEGKFAEEVVPVTIPQRKGDPIIFAEDEEYKAVNFDRLPTLPTVFKKEEGTVTAANASTLNDGASALVLVSKEKMEELGLKPLAKIISYADAAHEPENFTTAPSKALPIALKKAGLEVSDIDFFEFNEAFSVVGLANNKILGLDAAKVNVNGGAVALGHPLGSSGSRIIVTLINVLKQNNGKYGAAAICNGGGGASAIVIENI

SEQ ID NO: 11 - protein, 398 aa, Zygosaccharomyces rouxii
MSDNVYVVASCRTPVGSFQGSLASKTAIELGTAAVKGAISKVPQLKPETDYDEIIFGNVLSANSGQAPARQVALGAGLHNHIVDTTVNKVCASALRSIILGAQSIKCGTSDVVVAGGCESMTNTPYYMPAARSGARFGETKLVDGIQRDGLNDAYDGLAMGVHAEKCARDWDCSRQDQDEFAISSYQKAQKAQNEGKFDKEIVPITIKGFRGKPDTQVTKDEEPSKLNVEKLKSARTVFQKENGTVTAPNASPLNDGAAAVILVSEKKLKQLGLKPIAVIRGWGEAAHEPADFTWAPSLAVPKALTHAGIKDIGQIDFFEFNEAFSVVGLANTKILKIDPSKVNVYGGAVALGHPLGCSGARIVVTLLSVLEQENGKLGAAAICNGGGGASSIVLEKL

SEQ ID NO: 12 - protein, 391 aa, Pseudomonas pelagia
MSLNPRDVVIVDFGRTPMGRSKGGMYRNVRAENLSADLITGLLARNPKVDPAEVEDVIWGCVNQTLEQGWNVARMISLLTPIPHTSSAQTVSRLCGSSMSALHTAAQAIQTGNGDVFVVGGVEHMGHVGMMHGVDPNPSLSLYAAKASGMMGLTAEMLGKMHGISREQQDQFALRSHQLAHKATLNGGFKDEIIPMHGHDADGFLRVFSEDETIRPETTLESLAALRPAFNPKGGTVTAGSSSQITDGASCMIVMSAERAKALGLEPMAVIRSMAVAGVDPAIMGYGPVPATHKALKRAGMTMDNVDFVELNEAFAAQALPVLKDLKLLDKMEAKVNLHGGAIALGHPFGCSGARISGTLLNVMKQNGGTVGVSTMCIGLGQGITTVFERV

SEQ ID NO: 13 - protein, 400 aa, Thermus igniterrae
MPEAWIIQAVRTPIGKHGGALAPVRPDDLLAHALSALMERAGVPKEEVEDVYAGCANQAGEDNRNVARMALLLAGFPVEVAGCTVNRLCGSGLEAVAQAARAIWAGEGQVYVGAGVESMSRAPFVVPKAERPFPTGNLVMYDTTLGWRLVNPRMQALYGTESMGETAENLAEMYGIPREEQDRFALLSHQKAVRAWDEGRFPEEVVPVPVRRGKEEVLVAVDEGPRRDTSLEALAQLRPVFRQGGTVTAGNSSPLNDGAAALLLVSDAYARAHGLRPLARVRSVAVAGVPPRIMGIGPVPATRKALERAGLTLGDMGLIELNEAFAAQSLAVLREWGLSPEDPRLNPNGGAIALGHPLGASGARILTTLVYEMRRRGVPLGLATMCIGVGQGIAMVVEAV

SEQ ID NO: 14 - protein, 394 aa, Ralstonia solanacearum
MAREVVVVSGVRTAIGTFGGSLKDVAPCELGALVVREALARAQVKGEEVGHVVFGHVINTEPRDMYLSRVAALDGGVPAETPAFNVNRLCGSGLQAVVSAAQTILLGDVDVAIGGGAESMSRAPYLAPAARWGSRMGDAKLVDMMLGALHDPFHAIHMGVTAENVAREYGISRELQDQTALESHRRASRAIQAGYFKEQIVPVTIKSRKGDVQFDTDEHVRHDVTIEDMTRLKPVFAKENGTVTAGNASGLNDAAAALVLMERSLAEKRGLKPLARLVSYGHAGVDPNMMGIGPVPATRKALERAGLSVNDLDVVEANEAFAAQACAVTQQLGLDPAKVNPNGSGISLGHPIGATGALITVKALHELQRIGGRYALVTMCIGGGQGIAAVFERL

SEQ ID NO: 15 - protein, 394 aa, Citrobacter braakii
MKNCVIVSAARTAIGSFNGALATTGAVELGSIVIRAALERAHLDPQRVDEVIMGNVLQSGLGQNPARQALLKSGLSDTVCGFTVNKVCGSGLKSVALAALAIKAGQAQAIVAGGMENMSLAPYLLDAKARWGYRLGDGQLSDVILRDGLICAEHGYHMGITAENVAKEYGISREMQDELALQSQRKAVAAIESGAFEAEIVPVSVSSRKKTIIVERDEFPKADSTTEGLAALRPAFDKAGTVTAGNASGINDGAAALVIMEESAALEAGLKPLARIKAWASGGVAPAMMGMGPVPATLKALQLSGLQLSDIDLIEANEAFAAQFLAVGKTLGFDPQKVNVNGGAIALGHPIGASGARILVTLLHALQARDQTLGLATLCIGGGQGIAMIVERMN

SEQ ID NO: 16 - protein, 392 aa, Zoogloea ramigera
MSTPSIVIASARTAVGSFNGAFANTPAHELGATVISAVLERAGVAAGEVNEVILGQVLPAGEGQNPARQAAMKAGVPQEATAWGMNQLCGSGLRAVALGMQQIATGDASIIVAGGMESMSMAPHCAHLAGGVKMGDFKMIDTMIKDGLTDAFYGYHMGTTAENVAKQWQLSRDEQDAFAVASQNKAEAAQKDGRFKDEIVPFIVKGRKGDITVDADEYIRHGATLDSMAKLRPAFDKEGTVTAGNASGLNDGAAAALLMSEAEASRRGIQPLGRIVSWATVGVDPKVMGTGPIPASRKALERAGWKIGDLDLVEANEAFAAQACAVNKDLGWDPSIVNVNGGAIAIGHPIGASGARILNTLLFEMKRRGARKGLATLCIGGGMGVAMCIESL

SEQ ID NO: 17 - protein, 393 aa, Neorhizobium galegae
MSNPSIVIAAAGRTAVGSFNGAFANVPAHELGAAAIKGALARAGVEAAEVDEVILGQVLPAGEGQNPARQAAMKAGVPKEATAWGINQLCGSGLRAVALGMQQIALGDAKIIVAGGQESMSMAPHCGHLRGGVKMGDFKMIDTMIKDGLTDAFYGYHMGITAENIARQWQLSREEQDQFAVSSQNKAEAAQNAGRFKDEIIPYIIQTRKGDVTVENDEYIRAGATLDSMAKLRPAFDKEGTVTAGNASGLNDGAAAAVLMTEAEAARRGITPMVRIVSWATAGVDPQIMGTGPIPASRKALEKAGWKVGDLDLIEANEAFAAQSCAVTKDLGFDPSLVNVNGGAIAIGHPIGASGARILNTLLFEMKRRGAKKGLATLCIGGGMGVAMCLEAM

(19) United States
(12) Patent Application Publication
(10) Pub. No.: US 2020/0270653 A1
(43) Pub. Date: Aug. 27, 2020

(54) MODIFIED THIOLASES CAPABLE OF PRODUCING BRANCHED COMPOUNDS AND USES THEREOF

(71) Applicant: REPSOL, S.A., Madrid (ES)

(72) Inventors: Vicente BERNAL SÁNCHEZ, Móstoles (ES); Pamela TORRES SALAS, Móstoles (ES); Antonio MORREALE DE LEÓN, Móstoles (ES); Víctor BARRERA BURGOS, Móstoles (ES); María del Mar GONZÁLEZ BARROSO, Móstoles (ES); Fernando LÓPEZ GALLEGO, San Sebastián (ES); Federico GAGO BADENAS, Alcalá de Henares (ES); Pedro Alejandro SÁNCHEZ MURCIA, Alcalá de Henares (ES)

(73) Assignee: REPSOL, S.A., Madrid (ES)

(21) Appl. No.: 16/066,981

(22) PCT Filed: Dec. 29, 2016
(86) PCT No.: PCT/EP2016/082840

§ 371 Date: Jun. 28, 2018

(30) Foreign Application Priority Data
Dec. 29, 2015 (EP) 15382676.3

(51) Int. Cl. C12P 11/00; C12N 9/10

(52) U.S. Cl. CPC C12P 11/00; C12N 9/1029

(57) ABSTRACT

The invention relates to mutant thiolases capable of producing branched compounds which are considered of particular interest in industry. The invention also relates to method for obtaining said branched product.

### FIELD OF THE INVENTION

The invention relates to the field of bioengineering and particularly to enzymes with thiolase activity capable of generating precursors of 2-branched derivative compounds for industrial use.

### BACKGROUND OF THE INVENTION

Thiolases are a superfamily of enzymes catalyzing the transfer of a non-amino acid acyl group (for example an acetyl) by means of a Claisen condensation reaction to form a carbon-carbon bond. The thioester of a cysteine residue present in the active center thereof intervenes in the mechanism of reaction in all cases. These enzymes are involved in a number of metabolic pathways, including both biosynthesis (anabolic) and degradation (catabolic) pathways in eukaryotic and prokaryotic organisms.

The thiolase superfamily has been classified into three families according to functionality: (a) β-ketoacyfiacyl-carrier-protein synthases (KASs), (b) polyketide synthases (PKSs) and (c) the synthetic and degradative thiolase family. The two first groups are involved in fatty acid and complex polyketide metabolism, respectively. These two groups of proteins are mostly dependent on acyl carrier protein and involve carboxylation processes for substrate activation.

The third group is divided into two families: biosynthetic thiolases (EC 2.3.1.9) and degradative thiolases (EC 2.3.1.16), although both groups of enzymes are capable of catalyzing both the forward and reverse reactions under specific conditions. Although their substrate specificity is different, the thiolase superfamily conserves one and the same type of thiolase folding, in which each monomer is formed by two similar halves having a sandwich-type βαβαβαββ motif. In terms of their quaternary structure, they are usually organized as dimers or dimers of dimers in biological medium. In the latter, they are dimers of dimers interacting through four 136/137 loops, one for each monomer, and where the interactions are mostly hydrophobic-type interactions.

Enzymes belonging to the thiolase family have about 400 residues with an extensive loop of about 20 residues connecting two similar domains at the N- and C-terminal ends with βαβαβαββ folding. It is precisely this region which folds outwardly from the active center, forming a large part of the ligand interaction site. Catalytically relevant residues in these enzymes are located in the contiguous loops of the so-called α-3 helixes of respective N- and C-terminal domains (one histidine and two cysteines).

The general biosynthetic mechanism of reaction accepted for thiolases consists of two consecutive steps in which a covalent acylcysteine (Cys(Ac)) intermediate is generated (Scheme 1). Therefore, in a first step an acyl group (acetyl if substrate 1 is acetyl-CoA) is transferred to the catalytic cysteine (acceptor). In a second step a Claisen condensation step occurs between this catalytic cysteine and a new substrate 2 to obtain this substrate 2 acetylated in position 2.

(i) Acyl Transfer

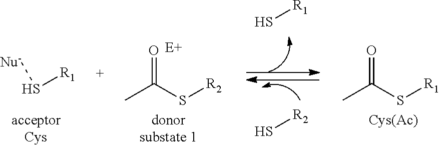

(ii) Claisen Condensation

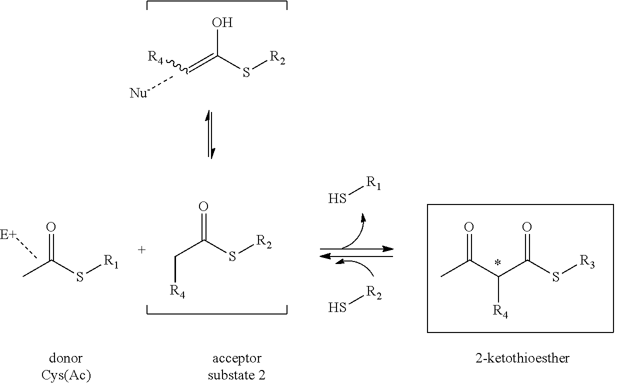

Scheme 1.—Mechanism of the Reaction Catalyzed by Thiolases.

The active center of thiolases is formed by two oxyanion holes (I and II) where the negative charges generated are stabilized throughout the catalytic cycle.

Compared to lineal products, branched products serve as a basis for obtaining a new family of compounds of industrial interest. In particular, branched products exhibit advantages as bio-fuels compared with the linear counterparts. However, most thiolases are not able to synthesize branched products.

Thus, there is a necessity to provide thiolases capable of synthesizing branched products to yield derivatives of interest in the industry.

### BRIEF DESCRIPTION OF THE INVENTION

In a first aspect, the invention relates to a mutant thiolase capable of catalyzing the formation of a compound of formula (I)

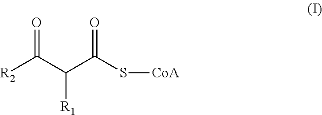

by the condensation of a compound of formula (II)

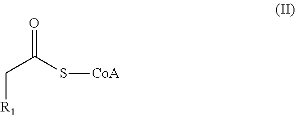

with a compound of formula (III)

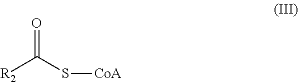

wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A, said mutant thiolase having at least a mutation at a position corresponding to position 293 in the polypeptide of SEQ ID NO: 1 wherein the thiolase shows at least a 75% sequence identity with a thiolase selected from the group consisting of of SEQ ID NO:1, 2, 3 or 4, with the proviso that said thiolase is not a thiolase consisting of the sequence SEQ ID NO: 7 or SEQ ID NO: 8.

In a second aspect the invention relates to a nucleic acid encoding a thiolase according to the first aspect of the invention.

In a third aspect, the invention relates to a vector comprising a nucleic acid according to the second aspect of the invention.

In a fourth aspect, the invention relates to a host cell comprising a nucleic acid according to the second aspect of the invention or a vector according to the third aspect of the invention.

In a fifth aspect, the invention relates to a process for the production of a compound of formula (I) comprising contacting a compound of formula (II) and a compound of formula (III) in the presence of a mutant thiolase capable of catalyzing the formation of a compound of formula (I)

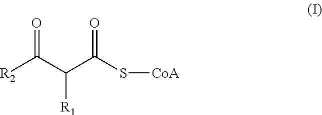

by the condensation of a compound of formula (II)

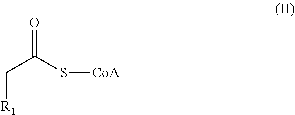

with a compound of formula (III)

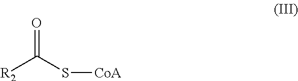

wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A, said mutant thiolase having at least a mutation with respect to the naturally-occurring thiolase, wherein said mutation is located at a position corresponding to position 293 in the polypeptide of SEQ ID NO: 1; and wherein said contacting is carried out under conditions adequate for the condensation of compounds of formula (II) and (III) into a compound of formula (I).

In a sixth aspect, the invention relates to a process for the production of a compound of formula (IV) comprising contacting a compound of formula (I) with a polypeptide having beta-ketoacyl-CoA reductase activity capable of catalyzing the formation of a compound of formula (IV)

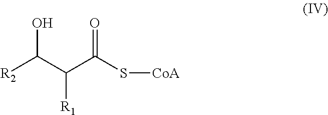

from the compound of formula (I), wherein R₁, R₂ and S-CoA are as defined in the first aspect of the invention.

### BRIEF DESCRIPTION OF THE DRAWINGS

FIG. 1. Alignment of amino acid sequences of thiolases from Saccharomyces cerevisiae (SEQ ID NO:1, Erg10), Flavobacterium bacteriaceae (SEQ ID NO:5, FbThl), Thermus thermophilus (SEQ ID NO:4, TtTlh), Ralstonia eutropha (SEQ ID NO:2, BktB) and Escherichia coli (SEQ ID NO:3, AtoB). Highly conserved regions are marked with an asterisk; colon indicates conservation between groups of strongly similar properties; period indicates conservation between groups of weakly similar properties. Amino acids with a key role in the catalytic cycle are framed

FIG. 2. (A) Yield of the reduction reaction of acetoacetyl-CoA and 3-keto-2-ethylbutyryl-CoA to 3-hydroxybutyryl-CoA (3HB-CoA) and 3-hydroxy-2-ethylbutyryl-CoA (3H2EB-CoA), respectively, as a function of PpFadB2 (SEQ ID NO:6) concentration. (B) Effect of PpFadB2 concentration on the stereoselectivity of the reduction reaction (expressed as [Peak2]/[Peak1] ratio) and on the production of the major “Peak 2” 3-hydroxy-2-ethylbutyryl-CoA isomer (expressed as [Peak2]/([Peak1]+[Peak2])·100). At the lowest protein concentration assayed (0.0001 mg/mL), only Peak2 isomer was detected.

FIG. 3. ¹H-NMR spectrum of 3-hydroxy-2-ethylbutyryl-CoA.

FIG. 4. Analysis of acetyl-CoA/butyryl-CoA condensation capacity of thiolases from Ralstonia eutropha (BktB). Total Ion Chromatogram (TIC) of (a) the standard mixture, (b) wild type BktB and (c) BktB_Var10. Peak legend: 1: 3H2EB-CoA Peak 1; 2: 3H2EB-CoA Peak 2; 3: 3HHB-CoA

FIG. 5. Comparison of 3HB-CoA production yields of thiolases from Saccharomyces cerevisiae (Erg10), Flavobacterium bacteriaceae (FbThl), Thermus thermophilus (TtTlhl), Ralstonia eutropha (BktB), Escherichia coli (AtoB) and their corresponding Var10 (triple mutants). Reactions were carried out at 37° C. in 100 mM MES buffer pH 6.0 using purified thiolase and FadB2 at a concentration of 0.19 and 0.15 mg/ml, respectively. Product formation was analyzed after 1 h and quantified by HPLC-MS. A) 3-hydroxy-2-ethylbutyryl-CoA yield, B) 3-hydroxyhexanoyl-CoA yield C) 3-hydroxybutyryl-CoA yield. D) Product selectivity of thiolase variants in acetyl-CoA/butyryl-CoA condensation assays.

FIG. 6. Comparison of variants of Erg10 thiolase from Saccharomyces cerevisiae. (SEQ ID NO:1) Reactions were carried out at 37° C. in 100 mM MES buffer pH 6.0 using purified thiolase and PpFadB2 at a concentration of 0.19 and 0.15 mg/ml, respectively. Product formation was analyzed and quantified by HPLC-MS after 1 h of reaction incubation.

FIG. 7. Product yields of thiolases from Saccharomyces cerevisiae (SEQ ID NO:1, Erg10) modified at position F293. Reactions were carried out at 37° C. in 100 mM MES buffer pH 6.0 using purified thiolases and PpFadB2 at a concentration of 0.19 and 0.15 mg/ml, respectively. Product formation was analyzed and quantified by HPLC-MS after 1 h of reaction incubation. (A) 3-hydroxy-2-ethylbutyryl-CoA (insert represents the product selectivity of selected mutants), (B) 3-hydroxybutyryl-CoA, (C) 3-hydroxyhexanoyl-CoA.

FIG. 8. Time course of product formation using (a) Erg10_F293D and (b) Erg10_F293A as biocatalyst. Reactions were carried out at 37° C. in 100 mM MES buffer pH 6.0 using purified thiolases and PpFadB2 at a concentration of 0.1 and 0.15 mg/ml, respectively. Product formation was quantified by HPLC-MS.

FIG. 9. A) LC-MS analysis of standard 3-hydroxy-pentanoyl-CoA (top), enzyme free control (middle) and Erg10-F293A reaction sample (bottom). B) MS analysis of peak 2 and peak 3.

FIG. 10. LC-MS analysis of enzyme free control (top) and Erg10-F293D reaction sample (bottom). In the insert, MS analysis of peak 4.

FIG. 11. Acetyl-CoA/acetyl-CoA condensation product yield of variants of Erg10 thiolase from Saccharomyces cerevisiae modified at position F293. Reactions were carried out at 37° C. in 100 mM MES buffer pH 6.0 using purified thiolases and PpFadB2 at a concentration of 0.19 and 0.15 mg/ml, respectively. Product formation was analyzed and quantified by HPLC-MS after 1 h of reaction incubation. 3-hydroxybutyryl-CoA product yields were normalized by comparison with the product yield of wild type Erg10.

FIG. 12. Effect of acetyl-CoA:butyryl-CoA ratio on the product profile of Erg10_F293D variant as biocatalyst after 1 h of reaction. Reactions were carried out at 37° C. in 100 mM MES buffer pH 6.0 using purified thiolases and PpFadB2 at a concentration of 0.19 and 0.15 mg/ml, respectively. Product formation quantified by HPLC-MS.

FIG. 13. Comparison of variants of BktB thiolase from Ralstonia eutropha (SEQ ID NO:2) Reactions were carried out at 37° C. in 100 mM MES buffer pH 6.0 using purified thiolase and PpFadB2 at a concentration of 0.19 and 0.15 mg/ml, respectively. Product formation was analyzed and quantified by HPLC-MS after 1 h of reaction incubation.

### DETAILED DESCRIPTION OF THE INVENTION

The inventors of the present invention have identified mutations in thiolase enzymes which improve their biosynthetic activity and, more in particular, are capable of generating branched products.

Based on these findings, the inventors have developed the processes of the present invention in their different embodiments that will be described now in detail.

### Mutant Thiolases of the Invention

In a first aspect, the invention relates to a mutant thiolase capable of catalyzing the formation of a compound of formula (I)

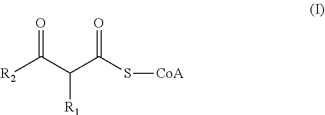

by the condensation of a compound of formula (II)

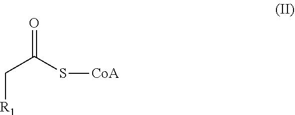

with a compound of formula (III)

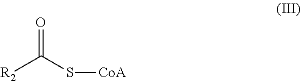

wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A, said mutant thiolase having at least a mutation at a position corresponding to position 293 in the polypeptide of SEQ ID NO: 1 wherein the thiolase shows at least a 75% sequence identity with a thiolase selected from the group consisting of SEQ ID NO:1, 2, 3 or 4, with the proviso that said thiolase is not a thiolase consisting of the sequence SEQ ID NO: 7 or SEQ ID NO: 8.

As it is used herein, the term “thiolase” refers to polypeptides capable of condensing 2 molecules comprising acetyl-CoA (AcCoA) to yield molecules comprising acetoacetyl-CoA. The term includes both, those thiolases which naturally catalyze the condensation of, for example, 2 acetyl-CoA molecules (biosynthetic thiolases, also known as acetoacetyl-CoA thiolase or acetyl-CoA acetyltransferase, with EC number 2.3.1.9) and thiolases which naturally catalyze the formation of acetyl-CoA from acetoacetyl-CoA and coenzyme A (degradative thiolases, also known as 3-ketoacyl-CoA thiolases or 3-oxoacyl-CoA thiolases, with EC number 2.3.1.16), but which can catalyze both the direct and the reverse reactions under suitable conditions. Preferred thiolases according to the present invention include, without limitation, Saccharomyces cerevisiae acetoacetyl-CoA thiolase (Erg10, SEQ ID NO: 1), Ralstonia eutropha acetyl-CoA acetyltransferase (BktB, SEQ ID NO: 2), Escherichia coli acetoacetyl-CoA thiolase (AtoB, SEQ ID NO: 3) and the Thermus thermophilus acetoacetyl-CoA thiolase (TtThl, SEQ ID NO: 4).

As it is used herein, the term “mutant” refers to any variant of a thiolase as defined above which is capable of condensing two molecules comprising acetyl-coenzyme A (AcCoA) with butyryl-coenzyme A (BuCoA) giving rise to the formation of 3-keto-2-ethylbutyryl-CoA), which differs from acetoacetyl-CoA thiolase due to the presence of an addition, substitution or deletion of one or more amino acids and which shows a sequence identity with native thiolase that is at least 75%, at least 80%, at least 85%, at least 90%, at least 95%, at least 96%, at least 97%, at least 98%, at least 99% or higher with respect to any of the sequences defined as SEQ ID NO:1 to 4. In a preferred embodiment, the sequence identity is determined over the entire length of the sequence. The person skilled in the art appreciates that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (e.g., GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley and Sons, Inc., (1995 Supplement)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively as well as the BLASTP algorithm (BLAST Manual, Altschul, S. et al., NCBI NLM NIH Bethesda, Md. 20894, Altschul, S., et al., J., 1990, Mol. Biol. 215:403-410). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score, T, when aligned with a word of the same length in a database sequence. T is referred to as, the neighbourhood word score threshold (Altschul et al, supra). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and percent sequence identity can employ the BESTFIT or GAP programs in the GCG Wisconsin Software package (Accelrys, Madison Wis.), using default parameters provided, or the multiple alignment program (available from the Swiss Institute of Bioinformatics, Lausanne, Switzerland), using, in some embodiments, the parameters below.

For purposes of the present disclosure, in some embodiments, the degree of percent amino acid sequence identity can be obtained by multiple alignment analysis, counting the number of identical matches in the alignment and dividing such number of identical matches by the length of the reference sequence, and using the following default parameters to achieve slow/accurate pairwise optimal alignments Output format: clustal w/o numbers; dealing input sequences: No; MBED-like clustering guide-tree: yes; Number of combined Iterations: default (0); Max guide tree iterations: default; max HMM iterations: default; Order: aligned.

The person skilled in the art will understand that the mutants according to the present invention showing a thiolase activity such as of acetyl-coenzyme A (AcCoA) condensation with butyryl-coenzyme A (BuCoA) can have reduced activity of acetyl-CoA condensation with respect to native sequences, such that the mutants according to the present invention can have a thiolase activity of acetyl-CoA condensation that is 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10% or less with respect to native thiolase activity.

Thiolase mutants according to the present invention have specific thiolase activity of condensing compounds of formulas (II) and (III) which is of at least 0.01, at least 0.05, at least 0.1, at least 0.5, at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95 or at least 100 mU/mg when the compounds of formula (II), preferably butyryl-coenzyme A (BuCoA), and the compounds of formula (III), preferably acetyl-CoA, are used as substrates.

As it is used herein, the term “C₁-C₆ alkyl group” refers to a linear- or branched-chain saturated non-cyclic hydrocarbon having 1 to 6 carbon atoms. Representative linear C₁-C₆ alkyl groups include, without limitation, methyl, ethyl, n-propyl, n-butyl, n-pentyl, n-hexyl; whereas branched C₁-C₆ alkyl groups include, without limitation, isopropyl, sec-butyl, isobutyl, tert-butyl, isopentyl, 2-methylbutyl, 3-methylbutyl, 2-methylpentyl, 3-methylpentyl, 4-methylpentyl, 2,3-dimethylbutyl, 2,2-dimethylbutyl, 3,3-dimethylbutyl and the like. Alkyl groups included in this invention can be optionally modified with one or more substituents.

As it is used herein, the twin “C₂-C₆ alkyl group” refers to a linear- or branched-chain saturated non-cyclic hydrocarbon having 2 to 6 carbon atoms. Representative linear C₂-C₆ alkyl groups include, without limitation, ethyl, n-propyl, n-butyl, n-pentyl, n-hexyl; whereas branched C₂-C₆ alkyl groups include, without limitation, isopropyl, sec-butyl, isobutyl, tert-butyl, isopentyl, 2-methylbutyl, 3-methylbutyl, 2-methylpentyl, 3-methylpentyl, 4-methylpentyl, 2,3-dimethylbutyl, 2,2-dimethylbutyl, 3,3-dimethylbutyl and the like. Alkyl groups included in this invention can be optionally modified with one or more substituents.

As it is used herein, the term “C₂-C₆ alkenyl group” refers to a linear- or branched-chain acyclic hydrocarbon having 2 to 6 carbon atoms and at least one carbon-carbon double bond. Representative linear and branched C₂-C₆ alkenyl groups include, without limitation, vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl and the like. The alkenyl groups can be optionally modified by one or more substituents.

As it is used herein, the term “C₂-C₆ alkynyl group” refers to a linear- or branched-chain acyclic hydrocarbon having 2 to 6 carbon atoms and at least one carbon-carbon triple bond. Representative linear and branched C₂-C₆ alkynyl groups include, without limitation, ethynyl, propynyl, butynyl, pentynyl, hexynyl and the like.

“Aryl” refers to aromatic hydrocarbon groups such as phenyl, naphthyl or anthracyl. The aryl group can be optionally substituted by one or more groups such as hydroxy, mercapto, halo, alkyl, phenyl, alkoxy, haloalkyl, nitro, cyano, carboxy, amino, dialkylamino, aminoalkyl, acyl and alkoxycarbonyl.

As it is used herein, “aryl-alkyl” is defined as an alkyl group as defined above associated with an aryl group as defined above.

As it is used herein, “benzyl” is defined as the —CH₂-phenyl group, wherein the phenyl group can be substituted or unsubstituted.

In a particular and preferred embodiment of the invention, R₁ is an ethyl group. In another particular and preferred embodiment of the invention, R₂ is a methyl group.

As it is used herein, the expression “position corresponding to position X in the polypeptide Y” refers to a position in the thiolase sequence coinciding with position X when said thiolase sequence overlaps with polypeptide Y sequence in a multiple sequence alignment including the mutant thiolase sequence, the polypeptide Y sequence and a variable number of sequences. In a preferred embodiment, the multiple sequence alignment is formed by the aforementioned thiolase sequences (sequences having SEQ ID NO:1 to 4). In a preferred embodiment, the multiple sequence alignment is carried out using at least 10 thiolase sequences, at least 15 sequences, at least 20 sequences, at least 25 sequences or more. The multiple sequence alignment can be obtained by using any standard method for that purpose as mentioned above and as it is shown in Example 1 of the present patent application. In a preferred embodiment, the multiple sequence alignment is carried out by means of the algorithm implemented in the CLUSTALW2 program (Larkin et al., Bioinformatics, 2007, 23:2947-2948) using standard parameters (Alignment type: slow; matrix: Gonnet; gap open: 10; gap extension: 0.1; KTUP: 1; Window length: 5; Score type: percent; Top Diags: 5 and Pair Gap: 3). In another preferred embodiment, the multiple sequence alignment is carried out by means of the algorithm implemented in the CLUSTAL OMEGA program (Sievers F. et al., Biology 7 Article number: 539 doi:10.1038/msb.2011.75) using standard parameters (HHalign algorithm with default parameters and the default transition matrix is Gonnet with a gap opening penalty of 6 bits and gap extension of 1 bit.

According to the present invention, the mutant thiolase has at least one mutation with respect to the naturally occurring thiolase wherein said mutation is located at a position corresponding to positions 293 in the polypeptide of SEQ ID NO: 1 with the proviso that said thiolase is not a thiolase consisting of the sequence SEQ ID NO: 7 or SEQ ID NO: 8.

In a particular embodiment of the invention, the sequence in said thiolase corresponding to the thiolase signature having the accession number PS00098 in the Prosite database has an identity of at least 35% to the sequence corresponding to said signature in the thiolase of SEQ ID NO:1, the sequence in said thiolase corresponding the thiolase signature having the accession number PS00737 in the Prosite database has an identity of at least 50% to the sequence corresponding to said signature in the thiolase of SEQ ID NO:1 and/or the sequence in said thiolase corresponding the thiolase signature having the accession number PS00099 in the Prosite database has an identity of at least 25% to the sequence corresponding to said signature in the thiolase of SEQ ID NO:1.

The expression “the sequence in said thiolase corresponding to the thiolase signature having the accession number X in the Prosite database” refers to the sequence of the thiolase of the invention coinciding with the signature X in the Prosite database in a sequence alignment including the mutant thiolase sequence of the invention (i.e. the thiolase comprising the sequences SEQs ID NO: 1-4) and the signature having the accession number X in the Prosite database. The sequence identity can be determined by using any suitable algorithm available to align two sequences previously detailed.

The signature having the accession number PS00098 is a conserved thiolase signature which has the following consensus pattern: [LIVM]-[NST]-{T}-x-C-[SAGLI]-[ST]-[SAG]-[LIVMFYNS]-x-[STAG]-[LIVM]-x(6)-[LIVM]. The sequence corresponding to signature having the PS00098 accession number in the Prosite database is found in the polypeptide of SEQ ID NO:1 at positions 87 to 105 and corresponds to the subsequence

VNKVCASAMKAIILGAQSI

The signature having the accession number PS00737 is a conserved thiolase signature which has the following consensus pattern: N-x (2)-G-G-x-[LIVM]-[SA]-x-G-H-P-x-[GAS]-x-[ST]-G. The sequence corresponding to signature having the PS00737 accession number in the Prosite database is found in the polypeptide of SEQ ID NO:1 at positions 344 to 360 and corresponds to the subsequence

NVYGGAVALGHPLGCSG

The signature having the accession number PS00099 is a conserved thiolase signature which has the following consensus pattern: [AG]-[LIVMA]-[STAGCLIVM]-[STAG]-[LIVMA]-C-{Q}-[AG]-x-[AG]-x-[AG]-x-[SAG] The sequence corresponding to signature having the PS00099 accession number in the Prosite database is found in the polypeptide of SEQ ID NO:1 at positions 379 to 392 and corresponds to the subsequence

GVAAICNGGGGASS

Thus, according with this embodiment, the sequence in the thiolase of the invention corresponding the thiolase signature having the accession number PS00098 in the Prosite database has an identity of at least 35% to the sequence corresponding to said thiolase signature of SEQ ID NO: 1. In a more particular embodiment, the sequence in the thiolase of the invention corresponding to the thiolase signature having the accession number PS00098 in the Prosite database has an identity of at, least 40% to the sequence corresponding to said thiolase signature of SEQ ID NO: 1. In another particular embodiment, said identity is of at least 45%, at least 50%, at least 55%, at least 60%, at least 65%, at least 70%, at least 75%, at least 80%, at least 85%, at least 90%, at least 95%, at least 99% or at least 100%. In a preferred embodiment, said sequence of the thiolase of the invention corresponding the thiolase signature having the accession number PS00098 in the Prosite database has an identity of about 37% to the sequence corresponding to said thiolase signature of SEQ ID NO: 1. In another preferred embodiment, said sequence of the thiolase of the invention corresponding the thiolase signature having the accession number PS00098 in the Prosite database has an identity of about 47% to the sequence corresponding to said thiolase signature of SEQ ID NO: 1. In a preferred embodiment, said sequence of the thiolase of the invention corresponding the thiolase signature having the accession number PS00098 in the Prosite database has an identity of about 58% to the sequence corresponding to said thiolase signature of SEQ ID NO: 1. In a preferred embodiment, said sequence of the thiolase of the invention corresponding the thiolase signature having the accession number PS00098 in the Prosite database has an identity of about 63% to the sequence corresponding to said thiolase signature of SEQ ID NO: 1.

Alternatively or additionally, the sequence of the thiolase of the invention corresponding the thiolase signature having the accession number PS00737 in the Prosite database has an identity of at least 50% to the sequence corresponding to said thiolase signature of SEQ ID NO: 1. Said signature having the accession number PS00737 is a conserved thiolase signature which has the following consensus pattern: N-x(2)-G-G-x-[LIVM]-[SA]-x-G-H-P-x-[GAS]-x-[ST]-G. In a more particular embodiment, said sequence of the thiolase of the invention corresponding the thiolase signature having the accession number PS00737 in the Prosite database has an identity of at least 55% to the sequence corresponding to said thiolase signature of SEQ ID NO: 1. In another particular embodiment, said identity is of at least 60%, at least 65%, at least 70%, at least 75%, at least 80%, at least 85%, at least 90%, at least 95%, at least 99% or at least 100%. In a preferred embodiment, said sequence of the thiolase of the invention corresponding the thiolase signature having the accession number PS00737 in the Prosite database has an identity of about 55% to the sequence corresponding to said thiolase signature of SEQ ID NO: 1. In another preferred embodiment, said sequence of the thiolase of the invention corresponding the thiolase signature having the accession number PS00737 in the Prosite database has an identity of about 76% to the sequence corresponding to said thiolase signature of SEQ ID NO: 1. In a preferred embodiment, said sequence of the thiolase of the invention corresponding the thiolase signature having the accession number PS00737 in the Prosite database has an identity of about 82% to the sequence corresponding to said thiolase signature of SEQ ID NO: 1. In a preferred embodiment, said sequence of the thiolase of the invention corresponding the thiolase signature having the accession number PS00737 in the Prosite database has an identity of about 86% to the sequence corresponding to said thiolase signature of SEQ ID NO: 1.

Alternatively or additionally, the sequence of the thiolase of the invention corresponding the thiolase signature having the accession number PS00099 in the Prosite database has an identity of at least 25% to the sequence corresponding to said thiolase signature of SEQ ID NO: 1. Said signature is a conserved thiolase signature which has the following consensus pattern: [AG]-[LIVMA]-[STAGCLIVM]-[STAG]-[LIVMA]-C-{Q}-[AG]-x-[AG]-x-[AG]-x-[SAG]. In a more particular embodiment, said sequence of the thiolase of the invention corresponding the thiolase signature having the accession number PS00099 in the Prosite database has an identity of at least 30% to the sequence corresponding to said thiolase signature of SEQ ID NO: 1. In another particular embodiment, said identity is of at least at least 35%, at least 40%, at least 45%, at least 50%, at least 55%, at least 60%, at least 65%, at least 70%, at least 75%, at least 80%, at least 85%, at least 90%, at least 95%, at least 99% or at least 100%. In a preferred embodiment, said sequence of the thiolase of the invention corresponding the thiolase signature having the accession number PS00099 in the Prosite database has an identity of about 35% to the sequence corresponding to said thiolase signature of SEQ ID NO: 1. In another preferred embodiment, said sequence of the thiolase of the invention corresponding the thiolase signature having the accession number PS00099 in the Prosite database has an identity of about 42% to the sequence corresponding to said thiolase signature of SEQ ID NO: 1. In a preferred embodiment, said sequence of the thiolase of the invention corresponding the thiolase signature having the accession number PS00099 in the Prosite database has an identity of about 82% to the sequence corresponding to said thiolase signature of SEQ ID NO: 1. In a preferred embodiment, said sequence of the thiolase of the invention corresponding the thiolase signature having the accession number PS00099 in the Prosite database has an identity of about 92% to the sequence corresponding to said thiolase signature of SEQ ID NO: 1.

In one embodiment, the mutant thiolase is selected from the group consisting of:
(i) The polypeptide of SEQ ID NO: 1, wherein the at least one mutation is located at position Phe293;
(ii) The polypeptide of SEQ ID NO: 2 wherein the at least one mutation is located at position Met290,
(iii) The polypeptide of SEQ ID NO: 3, wherein the at least one mutation is located at position Met289 and
(iv) The polypeptide of SEQ ID NO: 4, wherein the at least one mutation is located at position Met294.

In another embodiment, the mutant thiolase is selected from the group consisting of:
(i) The polypeptide of SEQ ID NO: 1, wherein the residue at position 293 is not Phe;
(ii) The polypeptide of SEQ ID NO: 2 wherein the residue at position 290 is not Met,
(iii) The polypeptide of SEQ ID NO: 3, wherein the residue at position 289 is not Met and
(iv) The polypeptide of SEQ ID NO: 4, wherein the residue at position 294 is not Met.

In another embodiment, the mutant thiolase is the polypeptide of SEQ ID NO: 1 wherein the at least one mutation located at position Phe293 is selected from the group consisting of Phe293Asp, Phe293Ala, Phe293Ser, Phe293Gly and Phe293Glu.

In another embodiment, the mutant thiolase is the polypeptide of SEQ ID NO: 2 wherein the at least one mutation at position Met290 is selected from the group consisting of Met290Asp, Met290Ala, Met290Ser, Met290Gly and Met290Glu.

In another embodiment, the mutant thiolase is the polypeptide of SEQ ID NO: 3, wherein at least one mutation located at position Met289 is selected from the group consisting of Met289Asp, Met289Ala, Met289Ser, Met289Gly and Met289Glu.

In another embodiment, the mutant thiolase is the polypeptide of SEQ ID NO: 4 wherein the at least one mutation located at position Met294 is selected from the group consisting of Met294Asp, Met294Ala, Met294Ser, Met294Gly, and Met294Glu.

In another embodiment, the thiolase according to the invention further comprises at least one additional mutation at a position selected from the group consisting of the residues corresponding to positions 90 and 383 in the polypeptide of SEQ ID NO: 1.

In one embodiment, the mutant thiolase is the polypeptide of SEQ ID NO: 1, containing mutations at positions Val90 and Phe293.

In one embodiment, the mutant thiolase is the polypeptide of SEQ ID NO: 1, containing mutations at positions Phe293 and Ile382.

In one embodiment, the mutant thiolase is the polypeptide of SEQ ID NO: 1, containing mutations at positions Val90, Phe293 and Ile383.

In one embodiment, the mutant thiolase is the polypeptide of SEQ ID NO: 1, containing mutations at positions Val90 and Phe293, wherein the mutations are such that the amino acid at position 90 is not Val and the amino acid at position 293 is not Phe.

In one embodiment, the mutant thiolase is the polypeptide of SEQ ID NO: 1, containing mutations at positions Phe293 and Ile382, wherein the mutations are such that the amino acid at position 293 is not Phe and the amino acid at position 382 is not Ile.

In one embodiment, the mutant thiolase is the polypeptide of SEQ ID NO: 1, containing mutations at positions Val90, Phe293 and Ile382, wherein the mutations are such that the amino acid at position 90 is not Val, the amino acid at position 293 is not Phe and the amino acid at position 382 is not Ile.

In one embodiment, the mutant thiolase is the polypeptide of SEQ ID NO: 1, containing mutations at positions Val90 and Phe293, wherein the mutation located at position Phe293 is selected from the group consisting of Phe293Asp, Phe293Ala, Phe293Ser, Phe293Gly and Phe293Glu and/or wherein the mutation located at position Val90 is a V90I mutation.

In one embodiment, the mutant thiolase is the polypeptide of SEQ ID NO: 1, containing mutations at positions Phe293 and Ile382 wherein the mutation located at position Phe293 is selected from the group consisting of Phe293Asp, Phe293Ala, Phe293Ser, Phe293Gly and Phe293Glu and/or wherein the mutation located at position Ile382 is a I383V mutation.

In one embodiment, the mutant thiolase is the polypeptide of SEQ ID NO: 1, containing mutations at positions Val90, Phe293 and Ile383, wherein the mutation located at position Phe293 is selected from the group consisting of Phe293Asp, Phe293Ala, Phe293Ser, Phe293Gly and Phe293Glu, wherein the mutation located at position Val90 is a V90I mutation and/or wherein the mutation located at position Ile382 is a I383V mutation.

In another embodiment, the mutant thiolase is selected from the group consisting of:
(i) The polypeptide of SEQ ID NO:1 containing the V90I and the F293A mutations,
(ii) The polypeptide of SEQ ID NO:1 containing the V90I and the F293D mutations,
(iii) The polypeptide of SEQ ID NO:1 containing the F293A and the I383V mutations,
(iv) The polypeptide of SEQ ID NO:1 containing the F293D and the I383V mutations,
(v) The polypeptide of SEQ ID NO:1 containing the V90I, the F293D and the I383V mutations and
(vi) The polypeptide of SEQ ID NO:1 containing the V90I, the F293A and the I383V mutations.

In another embodiment, the mutant thiolase is the thiolase of SEQ ID NO:2 containing mutations at positions Leu89 and Met290.

In another embodiment, the mutant thiolase is the thiolase of SEQ ID NO:2 containing mutations at positions Met290 and Met379.

In another embodiment, the mutant thiolase is the thiolase of SEQ ID NO:2 containing mutations at positions Leu89, Met290 and Met379.

In another embodiment, the mutant thiolase is the thiolase of SEQ ID NO:2 containing mutations at positions Leu89 and Met290, wherein the mutation is such that the amino acid at position 89 is not Leu and/or the amino acid at position 290 is not Met.

In another embodiment, the mutant thiolase is the thiolase of SEQ ID NO:2 containing mutations at positions Met290 and Met379, wherein the mutation is such that the amino acid at position 290 is not Met and the amino acid at position 379 is not Met.

In another embodiment, the mutant thiolase is the thiolase of SEQ ID NO:2 containing mutations at positions Leu89, Met290 and Met379, wherein the mutation is such that the amino acid at position 89 is not Leu, the amino acid at position 290 is not Met and/or the amino acid at position 379 is not Met.

In one embodiment, the mutant thiolase is the polypeptide of SEQ ID NO: 2, containing mutations at positions Leu89 and Met290, wherein the mutation located at position Met290 is selected from the group consisting of Met290Asp, Met290Ala, Met290Ser, Met290Gly and Met290Glu and/or the mutation at position Leu89 is a L89I mutation.

In one embodiment, the mutant thiolase is the polypeptide of SEQ ID NO: 2, containing mutations at positions Met290 and Met379, wherein the mutation located at position Met290 is selected from the group consisting of Met290Asp, Met290Ala, Met290Ser, Met290Gly and Met290Glu and/or the mutation at position Met379 is a Met379Val mutation.

In one embodiment, the mutant thiolase is the polypeptide of SEQ ID NO: 2, containing mutations at positions Leu89, Met290 and Met379, wherein the mutation located at position Met290 is selected from the group consisting of Met290Asp, Met290Ala, Met290Ser, Met290Gly and Met290Glu, the mutation at position Leu89 is a Leu89Ile mutation and/or the mutation at position Met379 is a Met379Val mutation.

In another embodiment, the mutant thiolase is selected from the group consisting of
(i) The polypeptide of SEQ ID NO:2 containing the L89I and the M290A mutations,
(ii) The polypeptide of SEQ ID NO:2 containing the L89I and the M290D mutations,
(iii) The polypeptide of SEQ ID NO:2 containing the M290D and the M379V mutations,
(iv) The polypeptide of SEQ ID NO:2 containing the M290A and the M379V mutations,
(v) The polypeptide of SEQ ID NO:2 containing the L89I, the M290A and the M379V mutations and
(vi) The polypeptide of SEQ ID NO:2 containing the L89I, the M290D and the M379V mutations.

In another embodiment, the mutant thiolase is the thiolase of SEQ ID NO:3 containing mutations at positions Val87 and Met289.

In another embodiment, the mutant thiolase is the thiolase of SEQ ID NO:3 containing mutations at positions Met289 and Leu378.

In another embodiment, the mutant thiolase is the thiolase of SEQ ID NO:3 containing mutations at positions Val87, Met289 and Leu378.

In another embodiment, the mutant thiolase is the thiolase of SEQ ID NO:3 containing mutations at positions Val87 and Met289, wherein the mutation is such that the amino acid at position 87 is not Val and/or the amino acid at position 289 is not Met.

In another embodiment, the mutant thiolase is the thiolase of SEQ ID NO:3 containing mutations at positions Met289 and Leu378, wherein the mutation is such that the amino acid at position 289 is not Met and the amino acid at position 378 is not Leu.

In another embodiment, the mutant thiolase is the thiolase of SEQ ID NO:3 containing mutations at positions Val87, Met289 and Met379, wherein the mutation is such that the amino acid at position 87 is not Val, the amino acid at position 289 is not Met and/or the amino acid at position 379 is not Met.

In another embodiment, the mutant thiolase is the thiolase of SEQ ID NO:3 containing mutations at positions Val87 and Met289, wherein the mutation at position 289 is Met289Asp, Met289Ala, Met289Ser, Met289Gly and Met289Glu and/or wherein the mutation at position Val87 is a Val87Ile mutation.

In another embodiment, the mutant thiolase is the thiolase of SEQ ID NO:3 containing mutations at positions Met289 and Leu378 wherein the mutation at position 289 is Met289Asp, Met289Ala, Met289Ser, Met289Gly and Met289Glu and/or wherein the mutation at position Leu378 is a Leu378Valmutation.

In another embodiment, the mutant thiolase is the thiolase of SEQ ID NO:3 containing mutations at positions Val87, Met289 and Leu378, wherein the mutation at position 289 is Met289Asp, Met289Ala, Met289Ser, Met289Gly and Met289Glu, wherein the mutation at position Val87 is a Val87Ile mutation and wherein the mutation at position Leu378 is a Leu378Val mutation.

In another embodiment, the mutant thiolase is selected from the group consisting of
(i), The polypeptide of SEQ ID NO:3 containing the V87I, the M289A and the L378V mutations and
(ii) the polypeptide of SEQ ID NO:3 containing the V87I, the M289D and the L378V mutations.

In another embodiment, the mutant thiolase is the thiolase of SEQ ID NO:4 containing mutations at positions Leu88 and Met294.

In another embodiment, the mutant thiolase is the thiolase of SEQ ID NO:4 containing mutations at positions Met294 and Met385.

In another embodiment, the mutant thiolase is the thiolase of SEQ ID NO:4 containing mutations at positions Leu88, Met294 and Met385.

In another embodiment, the mutant thiolase is the thiolase of SEQ ID NO:4 containing mutations at positions Leu88 and Met294, wherein the mutation is such that the amino acid at position 88 is not Leu and/or the amino acid at position 294 is not Met.

In another embodiment, the mutant thiolase is the thiolase of SEQ ID NO:4 containing mutations at positions Met294 and Met385, wherein the mutation is such that the amino acid at position 294 is not Met and the amino acid at position 385 is not Met.

In another embodiment, the mutant thiolase is the thiolase of SEQ ID NO:4 containing mutations at positions Leu88, Met294 and Met385, wherein the mutation is such that the amino acid at position 88 is not Leu, the amino acid at position 294 is not Met and the amino acid at position 385 is not Met.

In another embodiment, the mutant thiolase is the thiolase of SEQ ID NO:4 containing mutations at positions Leu88 and Met294, wherein the mutation located at position Met294 is selected from the group consisting of Met294Asp, Met294Ala, Met294Ser, Met294Gly, and Met294Glu and/or the mutation located at position Leu88 is a Leu88Ile mutation.

In another embodiment, the mutant thiolase is the thiolase of SEQ ID NO:4 containing mutations at positions Met294 and Met385, wherein the mutation located at position Met294 is selected from the group consisting of Met294Asp, Met294Ala, Met294Ser, Met294Gly, and Met294Glu and/or the mutation located at position Met385 is a Met385Val mutation.

In another embodiment, the mutant thiolase is the thiolase of SEQ ID NO:4 containing mutations at positions Leu88, Met294 and Met385, wherein the mutation located at position Met294 is selected from the group consisting of Met294Asp, Met294Ala, Met294Ser, Met294Gly, and Met294Glu, the mutation located at position Leu88 is a Leu88Ile mutation and/or the mutation located at position Met385 is a Met385Val mutation.

In another embodiment, the mutant thiolase is selected from the group consisting of
(i) The polypeptide of SEQ ID NO:4 containing the L88I, the M294A and the M385V mutations,
(ii) The polypeptide of SEQ ID NO:4 containing the L88I, the M294D and the M385V mutations,

In a second aspect, the invention relates to a nucleic acid encoding a thiolase according to the first aspect of the invention. The term “nucleic acid”, as used herein, refers to a deoxyribonucleotide or ribonucleotide polymer in either single or double stranded form and, unless otherwise limited, encompasses natural nucleotides and analogues of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in an oligonucleotide or in a polynucleotide. A “nucleotide sequence” or “nucleic acid sequence” refers to the sequence of bases in an oligonucleotide or in a polynucleotide.

The term polynucleotide refers to a sequence of at least 10 nucleotides, preferably at least 16 nucleotides, preferably at least 20 nucleotides, preferably at least 25 nucleotides, preferably at least 100 nucleotides or more, including both polyribonucleotides and polydeoxyribonucleotides or combinations thereof. The term “oligonucleotide” also refers to molecules formed by conventional nucleotides bound by conventional phosphodiester bonds as well as variants thereof, including modifications in the purine or pyrimidine residues and modifications in the ribose or deoxyribose residues designed for increasing biological stability of the oligonucleotide or for stabilizing the physical stability of the hairpin-shaped structure. Examples of modified nucleotides that can be used in the present invention include, but are not limited to, nucleotides having at position 2′ of the sugar a substituent selected from the fluoro, hydroxyl, amino, azido, alkyl, alkoxy, alkoxyalkyl, methyl, ethyl, propyl, butyl group or a functionalized alkyl group such as ethylamino, propylamino and butylamino. Alternatively, the alkoxy group is methoxy, ethoxy, propoxy or a functionalized alkoxy group according to the formula —O(CH₂)q-R, where q is 2 to 4 and R is an amino, methoxy or ethoxy group. Suitable alkoxyalkyl groups are methoxyethyl and ethoxyethyl. Oligonucleotides in which different nucleotides contain different modifications at position 2′ are also part of the invention. Alternatively or additionally, the oligonucleotides of the invention can contain modified bonds such as phosphodiester-, phosphotriester-, phosphorothioate-, phosphorodithioate-, phosphoroselenoate-, phosphorodiselenoate-, phosphoroanilothioate-, phosphoramidate-, methylphosphonate-, boranephosphonate-type bonds as well as combinations thereof, or they are peptide nucleic acids (PNAs), in which the different nucleotides are bound by amide bonds.

In a third aspect, the invention relates to a vector comprising a nucleic acid according to the second aspect of the invention. The term “vector”, as used herein, refers to a construct capable of delivering, and preferably additionally expressing, one or more polynucleotides of interest into a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. This term also relates to targeting constructs which allow for random or site-directed integration of the targeting construct into genomic DNA. Such targeting constructs, preferably, comprise DNA of sufficient length for either homologous recombination or heterologous integration.

In a fourth aspect, the invention relates to a host cell comprising a nucleic acid according to the second aspect of the invention or a vector according to the third aspect of the invention. Transformation of a host cell with recombinant DNA can be carried out by conventional techniques that are well known to those of ordinary skill in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding the mutant thiolase of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. See Gluzman Y, Ed., “Eukaryotic Viral Vectors” (Cold Spring Harbor Laboratory Press, Cold Spring Harbor, N.Y., US, 1982).

### In Vitro Process for the Production of the Compound of Formula (I)

In a fifth aspect, the invention relates to a process, hereinafter “the first process of the invention” for the production of a compound of formula (I) comprising contacting a compound of formula (II) and a compound of formula (III) in the presence of a mutant thiolase capable of catalyzing the formation of a compound of formula (I)

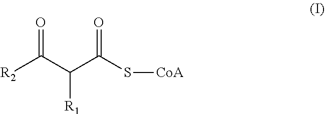

by the condensation of a compound of formula (II)

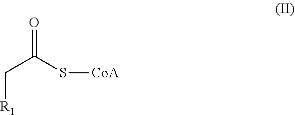

with a compound of formula (III)

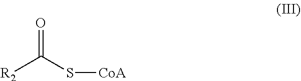

wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A, said mutant thiolase having at least a mutation with respect to the naturally-occurring thiolase, wherein said mutation is located at a position corresponding to position 293 in the polypeptide of SEQ ID NO: 1; and wherein said contacting is carried out under conditions adequate for the condensation of compounds of formula (II) and (III) into a compound of formula (I).

The terms “mutant”, “thiolase”, “compound of formula I”, “compound of formula II”, and “compound of formula III” and their particulars have been defined in the context of the first embodiment of the invention.

In a particular embodiment of the first process of the invention, the sequence in said thiolase corresponding the thiolase signature having the accession number PS00098 in the Prosite database has an identity of at least 35% to the sequence corresponding to said signature in the thiolase of SEQ ID NO:1, the sequence in said thiolase corresponding the thiolase signature having the accession number PS00737 in the Prosite database has an identity of at least 50% to the sequence corresponding to said signature in the thiolase of SEQ ID NO:1 and/or the sequence in said thiolase corresponding the thiolase signature having the accession number PS00099 in the Prosite database has an identity of at least 25% to the sequence corresponding to said signature in the thiolase of SEQ ID NO:1.

In one embodiment, the mutant thiolase is selected from the group consisting of:
(i) The polypeptide of SEQ ID NO: 1, wherein the at least one mutation is located at position Phe293;
(ii) The polypeptide of SEQ ID NO: 2 wherein the at least one mutation is located at position Met290,
(iii) The polypeptide of SEQ ID NO: 3, wherein the at least one mutation is located at position Met289 and
(iv) The polypeptide of SEQ ID NO: 4, wherein the at least one mutation is located at position Met294.

In another embodiment, the mutant thiolase is selected from the group consisting of:
(i) The polypeptide of SEQ ID NO: 1, wherein the residue at position 293 is not Phe;
(ii) The polypeptide of SEQ ID NO: 2 wherein the residue at position 290 is not Met,
(iii) The polypeptide of SEQ ID NO: 3, wherein the residue at position 289 is not Met and
(iv) The polypeptide of SEQ ID NO: 4, wherein the residue at position 294 is not Met.

In another embodiment, the mutant thiolase is the polypeptide of SEQ ID NO: 1 wherein the at least one mutation located at position Phe293 is selected from the group consisting of Phe293Asp, Phe293Ala, Phe293Ser, Phe293Gly and Phe293Glu.

In another embodiment, the mutant thiolase is the polypeptide of SEQ ID NO: 2 wherein the at least one mutation at position Met290 is selected from the group consisting of Met290Asp, Met290Ala, Met290Ser, Met290Gly and Met290Glu.

In another embodiment, the mutant thiolase is the polypeptide of SEQ ID NO: 3, wherein at least one mutation located at position Met289 is selected from the group consisting of Met289Asp, Met289Ala, Met289Ser, Met289Gly and Met289Glu.

In another embodiment, the mutant thiolase is the polypeptide of SEQ ID NO: 4 wherein the at least one mutation located at position Met294 is selected from the group consisting of Met294Asp, Met294Ala, Met294Ser, Met294Gly, and Met294Glu.

In another embodiment, the thiolase according to the invention further comprises at least one additional mutation at a position selected from the group consisting of the residues corresponding to positions 90 and 383 in the polypeptide of SEQ ID NO: 1.

In one embodiment, the mutant thiolase is the polypeptide of SEQ ID NO: 1, containing mutations at positions Val90 and Phe293.

In one embodiment, the mutant thiolase is the polypeptide of SEQ ID NO: 1, containing mutations at positions Phe293 and Ile382.

In one embodiment, the mutant thiolase is the polypeptide of SEQ ID NO: 1, containing mutations at positions Val90, Phe293 and Ile383.

In one embodiment, the mutant thiolase is the polypeptide of SEQ ID NO: 1, containing mutations at positions Val90 and Phe293, wherein the mutations are such that the amino acid at position 90 is not Val and the amino acid at position 293 is not Phe.

In one embodiment, the mutant thiolase is the polypeptide of SEQ ID NO: 1, containing mutations at positions Phe293 and Ile382, wherein the mutations are such that the amino acid at position 293 is not Phe and the amino acid at position 382 is not Ile.

In one embodiment, the mutant thiolase is the polypeptide of SEQ ID NO: 1, containing mutations at positions Val90, Phe293 and Ile382, wherein the mutations are such that the amino acid at position 90 is not Val, the amino acid at position 293 is not Phe and the amino acid at position 382 is not Ile.

In one embodiment, the mutant thiolase is the polypeptide of SEQ ID NO: 1, containing mutations at positions Val90 and Phe293, wherein the mutation located at position Phe293 is selected from the group consisting of Phe293Asp, Phe293Ala, Phe293Ser, Phe293Gly and Phe293Glu and/or wherein the mutation located at position Val90 is a Val90Ile mutation.

In one embodiment, the mutant thiolase is the polypeptide of SEQ ID NO: 1, containing mutations at positions Phe293 and Ile382 wherein the mutation located at position Phe293 is selected from the group consisting of Phe293Asp, Phe293Ala, Phe293Ser, Phe293Gly and Phe293Glu and/or wherein the mutation located at position Ile382 is a leI383Valmutation.

In one embodiment, the mutant thiolase is the polypeptide of SEQ ID NO: 1, containing mutations at positions Val90, Phe293 and Ile383, wherein the mutation located at position Phe293 is selected from the group consisting of Phe293Asp, Phe293Ala, Phe293Ser, Phe293Gly and Phe293Glu, wherein the mutation located at position Val90 is a Val90Ile mutation and/or wherein the mutation located at position Ile382 is a Ile383Valmutation.

In another embodiment, the mutant thiolase is selected from the group consisting of:
(i) The polypeptide of SEQ ID NO:1 containing the V90I and the F293A mutations,
(ii) The polypeptide of SEQ ID NO:1 containing the V90I and the F293D mutations,
(iii) The polypeptide of SEQ ID NO:1 containing the F293A and the I383V mutations,
(iv) The polypeptide of SEQ ID NO:1 containing the F293D and the I383V mutations,
(v) The polypeptide of SEQ ID NO:1 containing the V90I, the F293D and the I383V mutations and
(vi) The polypeptide of SEQ ID NO:1 containing the V90I, the F293A and the I383V mutations.

In another embodiment, the mutant thiolase is the thiolase of SEQ ID NO:2 containing mutations at positions Leu89 and Met290.

In another embodiment, the mutant thiolase is the thiolase of SEQ ID NO:2 containing mutations at positions Met290 and Met379.

In another embodiment, the mutant thiolase is the thiolase of SEQ ID NO:2 containing mutations at positions Leu89, Met290 and Met379.

In another embodiment, the mutant thiolase is the thiolase of SEQ ID NO:2 containing mutations at positions Leu89 and Met290, wherein the mutation is such that the amino acid at position 89 is not Leu and/or the amino acid at position 290 is not Met.

In another embodiment, the mutant thiolase is the thiolase of SEQ ID NO:2 containing mutations at positions Met290 and Met379, wherein the mutation is such that the amino acid at position 290 is not Met and the amino acid at position 379 is not Met.

In another embodiment, the mutant thiolase is the thiolase of SEQ ID NO:2 containing mutations at positions Leu89, Met290 and Met379, wherein the mutation is such that the amino acid at position 89 is not Leu, the amino acid at position 290 is not Met and/or the amino acid at position 379 is not Met.

In one embodiment, the mutant thiolase is the polypeptide of SEQ ID NO: 2, containing mutations at positions Leu89 and Met290, wherein the mutation located at position Met290 is selected from the group consisting of Met290Asp, Met290Ala, Met290Ser, Met290Gly and Met290Glu and/or the mutation at position Leu89 is a Leu89Ile mutation.

In one embodiment, the mutant thiolase is the polypeptide of SEQ ID NO: 2, containing mutations at positions Met290 and Met379, wherein the mutation located at position Met290 is selected from the group consisting of Met290Asp, Met290Ala, Met290Ser, Met290Gly and Met290Glu and/or the mutation at position Met379 is a Met379Val mutation.

In one embodiment, the mutant thiolase is the polypeptide of SEQ ID NO: 2, containing mutations at positions Leu89, Met290 and Met379, wherein the mutation located at position Met290 is selected from the group consisting of Met290Asp, Met290Ala, Met290Ser, Met290Gly and Met290Glu, the mutation at position Leu89 is a Leu89Ile mutation and/or the mutation at position Met379 is a Met379Val mutation.

In another embodiment, the mutant thiolase is selected from the group consisting of
(i) The polypeptide of SEQ ID NO:2 containing the L89I and the M290A mutations,
(ii) The polypeptide of SEQ ID NO:2 containing the L89I and the M290D mutations,
(iii) The polypeptide of SEQ ID NO:2 containing the M290D and the M379V mutations,
(iv) The polypeptide of SEQ ID NO:2 containing the M290A and the M379V mutations,
(v) The polypeptide of SEQ ID NO:2 containing the L89I, the M290A and the M379V mutations and
(vi) The polypeptide of SEQ ID NO:2 containing the L89I, the M290D and the M379V mutations.

In another embodiment, the mutant thiolase is the thiolase of SEQ ID NO:3 containing mutations at positions Val87 and Met289.

In another embodiment, the mutant thiolase is the thiolase of SEQ ID NO:3 containing mutations at positions Met289 and Leu378.

In another embodiment, the mutant thiolase is the thiolase of SEQ ID NO:3 containing mutations at positions Val87, Met289 and Leu378.

In another embodiment, the mutant thiolase is the thiolase of SEQ ID NO:3 containing mutations at positions Val87 and Met289, wherein the mutation is such that the amino acid at position 87 is not Val and/or the amino acid at position 289 is not Met.

In another embodiment, the mutant thiolase is the thiolase of SEQ ID NO:3 containing mutations at positions Met289 and Leu378, wherein the mutation is such that the amino acid at position 289 is not Met and the amino acid at position 378 is not Leu.

In another embodiment, the mutant thiolase is the thiolase of SEQ ID NO:3 containing mutations at positions Val87, Met289 and Met379, wherein the mutation is such that the amino acid at position 87 is not Val, the amino acid at position 289 is not Met and/or the amino acid at position 379 is not Met.

In another embodiment, the mutant thiolase is the thiolase of SEQ ID NO:3 containing mutations at positions Val87 and Met289, wherein the mutation at position 289 is Met289Asp, Met289Ala, Met289Ser, Met289Gly and Met289Glu and/or wherein the mutation at position Val87 is a Val87Ile mutation.

In another embodiment, the mutant thiolase is the thiolase of SEQ ID NO:3 containing mutations at positions Met289 and Leu378 wherein the mutation at position 289 is Met289Asp, Met289Ala, Met289Ser, Met289Gly and Met289Glu and/or wherein the mutation at position Leu378 is a Leu378Valmutation.

In another embodiment, the mutant thiolase is the thiolase of SEQ ID NO:3 containing mutations at positions Val87, Met289 and Leu378, wherein the mutation at position 289 is Met289Asp, Met289Ala, Met289Ser, Met289Gly and Met289Glu, wherein the mutation at position Val87 is a Val87Ile mutation and wherein the mutation at position Leu378 is a Leu378Valmutation.

In another embodiment, the mutant thiolase is selected from the group consisting of
(i) The polypeptide of SEQ ID NO:3 containing the V87I, the M289A and the L378V mutations and
(ii) the polypeptide of SEQ ID NO:3 containing the V87I, the M289D and the L378V mutations.

In another embodiment, the mutant thiolase is the thiolase of SEQ ID NO:4 containing mutations at positions Leu88 and Met294.

In another embodiment, the mutant thiolase is the thiolase of SEQ ID NO:4 containing mutations at positions Met294 and Met385.

In another embodiment, the mutant thiolase is the thiolase of SEQ ID NO:4 containing mutations at positions Leu88, Met294 and Met385.

In another embodiment, the mutant thiolase is the thiolase of SEQ ID NO:4 containing mutations at positions Leu88 and Met294, wherein the mutation is such that the amino acid at position 88 is not Leu and/or the amino acid at position 294 is not Met.

In another embodiment, the mutant thiolase is the thiolase of SEQ ID NO:4 containing mutations at positions Met294 and Met385, wherein the mutation is such that the amino acid at position 294 is not Met and the amino acid at position 385 is not Met.

In another embodiment, the mutant thiolase is the thiolase of SEQ ID NO:4 containing mutations at positions Leu88, Met294 and Met385, wherein the mutation is such that the amino acid at position 88 is not Leu, the amino acid at position 294 is not Met and the amino acid at position 385 is not Met.

In another embodiment, the mutant thiolase is the thiolase of SEQ ID NO:4 containing mutations at positions Leu88 and Met294, wherein the mutation located at position Met294 is selected from the group consisting of Met294Asp, Met294Ala, Met294Ser, Met294Gly, and Met294Glu and/or the mutation located at position Leu88 is a Leu88Ile mutation.

In another embodiment, the mutant thiolase is the thiolase of SEQ ID NO:4, containing mutations at positions Met294 and Met385, wherein the mutation located at position Met294 is selected from the group consisting of Met294Asp, Met294Ala, Met294Ser, Met294Gly, and Met294Glu and/or the mutation located at position Met385 is a Met385Val mutation.

In another embodiment, the mutant thiolase is the thiolase of SEQ ID NO:4 containing mutations at positions Leu88, Met294 and Met385, wherein the mutation located at position Met294 is selected from the group consisting of Met294Asp, Met294Ala, Met294Ser, Met294Gly, and Met294Glu, the mutation located at position Leu88 is a L88I mutation and/or the mutation located at position Met385 is a Met385Val mutation.

In another embodiment, the mutant thiolase is selected from the group consisting of:
(i) The polypeptide of SEQ ID NO:4 containing the L88I, the M294A and the M385V mutations,
(ii) The polypeptide of SEQ ID NO:4 containing the L88I, the M294D and the M385V mutations,

The expression “contacting a compound of formula (II) and a compound of formula (III) in the presence of a thiolase”, as used herein, refers to the incubation of said thiolase and said compounds of formula (II) and (III) for the interaction between said compounds and said thiolase; thereby leading to the formation of the compound of formula (I).

The conditions required for the thiolase to condense the compound of formula (II) and the compound of formula (III) producing the compound of formula (I) include an appropriate ratio between the thiolase and the substrates in terms of concentration and appropriate conditions of salts, cofactors, pH and temperature as well to an appropriate reaction time.

Suitable salt conditions for carrying the first process according to the invention include, without limitation, any buffer commonly known such as phosphate buffer, a bicarbonate buffer, a carbonate buffer, a Tris buffer, a borate buffer, a succinate buffer, a histidine buffer, a citrate buffer or a 2-(N-morpholino)ethanesulphonic acid buffer (MES) at concentrations of at least 10 mM, at least 20 mM, at least 30 mM, at least 40 mM, at least 50 mM, at least 60 mM, at least 70 mM, at least 80 mM, at least 90 mM, at least 100 mM, at least 200 mM, at least 300 mM or more. In a preferred embodiment, the process according to the present invention is carried out in 100 mM MES buffer.

Suitable pH values for carrying the first process according to the invention include, without limitation, values of 4.5, 5, 5.5, 6, 6.5, 7, 7.5, 8, 8.5, 9 and 9.5. In a preferred embodiment, the process is carried out at a pH of about 6.

Suitable temperature conditions for carrying the first process according to the invention include, without limitation, about 20° C., about 25° C., about 30° C., about 35° C., about 37° C. In a preferred embodiment, the process is carried out at 37° C.

Suitable reaction times for carrying the process according to the invention include, without limitation, at least 1 ms, at least 1 s, at least 15 s, at least 30 s, at least 1 min, at least 2 min, at least 3 min, at least 4 min, at least 5 min, at least 6 min, at least 7 min, at least 8 min, at least 9 min, at least 10 min, at least 15 min, at least 20 min, at least 25 min, at least 30 min, at least 35 min, at least 40 min, at least 45 min, at least 50 min, at least 55 min, at least 1 h, at least 2 h, at least 3 h, at least 4 h, at least 5 h, at least 6 h, at least 7 h, at least 8 h, at least 9 h, at least 10 h or more. In a preferred embodiment, the reaction time is 1 hour.

The ratio of the concentration of compounds (II) and (III) in the first process according to the invention is not particularly limiting. Accordingly, suitable ratios of the concentration of compounds (II) to compounds (III) are 1000:1, 500:1, 100:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:4, 1:5, 1:6, 1:7, 1:8, 1:9, 1:10, 1:20, 1:30, 1:40, 1:50, 1:100, 1:500 or 1:1000. In one embodiment, both compounds are used at equimolar concentrations. Suitable concentrations of the compounds (II) and (III) include, without limitation, about 1 nM, about 5 nM, about 10 nM, about 20 nM, about 30 nM, about 40 nM, about 50 nM, about 100 nM, about 500 nM, about 1 μM, about 10 μM, about 20 μM, about 30 μM, about 40 μM, about 50 μM, about 100 μM, about 500 μM, about 1 mM, about 5 mM, about 10 mM, about 20 mM, about 30 mM, about 40 mM, about 50 mM, about 100 mM or more. In a preferred embodiment, the concentration of substrates (I) and (II) is between 0.1 and 1 mM.

Suitable concentrations of the thiolase for carrying the first process according to the invention include, without limitation, about 1 μg/mL, about 2 μg/mL, about 3 μg/mL, about 5 μg/mL, about 8 μg/mL, about 10 μg/mL, about 20 μg/mL, about 30 μg/mL, about 50 μg/mL, about 75 μg/mL, about 100 μg/mL, about 200 μg/mL, about 300 μg/mL, about 500 μg/mL, about 750 μg/mL, about 1 mg/mL, about 2 mg/mL, about 3 mg/mL, about 5 mg/mL, about 7.5 mg/mL, about 10 mg/mL, about 20 mg/mL, about 30 mg/mL, about 50 mg/mL, about 75 mg/mL, about 100 mg/mL. In a preferred embodiment, the concentration of the thiolase is between 0.05 and 1 mg/mL.

In a particular and preferred embodiment of the invention, the first process of the invention further comprises contacting the compound of formula (I) with a polypeptide having beta-ketoacyl-CoA reductase activity capable of catalyzing the formation of a compound of formula (IV)

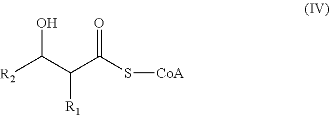

from the compound of formula (I) and wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A.

The term “polypeptide having beta-ketoacyl-CoA reductase activity”, as used herein refers to a polypeptide which catalyzes the NADP(H) conversion of branched aldehydes or ketones to their respective alcohols, wherein the term NAD(P)H refers to either NADH or NADPH as the cofactor. The polypeptide having beta-ketoacyl-CoA reductase activity for use in the present invention is a biological catalyst; thus the polypeptide having beta-ketoacyl-CoA reductase activity for use in the invention is an enzyme.

The beta-ketoacyl-CoA reductase activity can be determined by any method known in the art suitable for the determination of the activity of NAD(P)H-dependent aldehyde reductase activity such as liquid chromatography coupled to mass spectrometry or spectrophotometrically by monitoring the oxidation of NAD(P)H at 240 nm.

Polypeptide having beta-ketoacyl-CoA reductase activity for use in the present invention include polypeptides having specific activities in the range of at least 0.001, at least 0.01, at least 0.1, at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 mU/mg, at least 200 mU/mg, at least 400 mU/mg, at least 800 mU/mg, at least 1000 mU/mg or more when using 3-keto-2-ethyl-butyryl-CoA as substrate.

According to the present invention, the sequence of a polypeptide having beta-ketoacyl-CoA reductase activity has an identity of at least 45% to the sequence of SEQ ID NO: 6, wherein SEQ ID NO: 6 corresponds to the amino acid sequence of the beta-ketoacyl-CoA reductase from Pseudomonas putida.

In a particular embodiment, the sequence of a polypeptide having beta-ketoacyl-CoA reductase activity has an identity of at least 46%, at least 47%, at least 48%, at least 49%, at least 50%, at least 51%, at least 52%, at least 53%, at least 54%, at least 55%, at least 60%, at least 65%, at least 70%, at least 75%, at least 80%, at least 85%, at least 90%, at least 95%, at least 99% or more to the sequence SEQ ID NO:6. In a preferred embodiment, the above identity value is determined over the complete length of the polypeptides being compared. The term “identity” as well as suitable algorithms for determining the percentage of identity among two sequences have been detailed above.

In a particular and preferred embodiment of the invention, the polypeptide having beta-ketoacyl-CoA reductase activity consists of the amino acid sequence of the beta-ketoacyl-CoA reductase protein from Pseudomonas putida and which is shown in SEQ ID NO: 6.

As the person skilled in the art will understand, this particular embodiment includes suitable conditions for carrying out the reaction by which the compound (IV) is obtained from compound (I). Said conditions include an appropriate ratio between the polypeptide having beta-ketoacyl-CoA reductase activity and the substrate in terms of concentration and appropriate conditions of salts, cofactors, pH and temperature as well to an appropriate reaction time.

Suitable salt, pH, temperature conditions, suitable concentration of both cofactor and compound (I) and appropriate reaction time have been detailed above.

Suitable concentrations of the polypeptide having beta-ketoacyl-CoA reductase activity include, without limitation, about 1 μg/mL, about 2 μg/mL, about 3 tag/mL, about 5 μg/mL, about 8 μg/mL, about 10 μg/mL, about 20 μg/mL, about 30 μg/mL, about 50 μg/mL, about 75 μg/mL, about 100 μg/mL, about 200 μg/mL, about 300 μg/mL, about 500 μg/mL, about 750 μg/mL, about 1 mg/mL, about 2 mg/mL, about 3 mg/mL, about 5 mg/mL, about 7.5 mg/mL, about 10 mg/mL, about 20 mg/mL, about 30 mg/mL, about 50 mg/mL, about 75 mg/mL, about 100 mg/mL. In a preferred embodiment, the concentration of the polypeptide having beta-ketoacyl-CoA reductase activity is between 0.05 and 1 mg/mL.

In a particular and preferred embodiment of the first process of the invention, R₁ is an ethyl group. In another particular and preferred embodiment of the first process invention, R₂ is a methyl group.

### In Vitro Process for the Production of the Compound of Formula (IV)

In a seventh aspect, the invention relates to a process for the production of a compound of formula (IV), hereinafter “the third process of the invention”, comprising contacting a compound of formula (I) with a polypeptide having beta-ketoacyl-CoA reductase activity capable of catalyzing the formation of a compound of formula (IV)

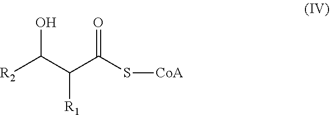

from the compound of formula (I), wherein R₁ and R₂ are independently selected from the group consisting of linear or branched C₂-C₆ alkyl, linear or branched C₂-C₆ alkenyl, linear or branched C₂-C₆ alkynyl, aryl, aryl-alkyl and benzyl, and S-CoA is coenzyme A.

The terms “polypeptide having beta-ketoacyl-CoA reductase activity”, “compound of formula I”, “compound of formula IV”, “C₁-C₆ alkyl”, “C₂-C₆ alkyl”, “C₂-C₆ alkenyl”, “C₂-C₆ alkynyl”, “aryl”, “aryl-alkyl”, “benzyl” as the particulars thereof have been previously defined and are used herein with the same meaning.

The expression “contacting a compound of formula (I) with a polypeptide having beta-ketoacyl-CoA reductase activity”, as used herein, refers to the incubation of said polypeptide and said compound of formula (I) for the interaction between said compound and said polypeptide; thereby leading the formation of the compound of formula (IV).

As the person skilled in the art will understand, this process is carried out under suitable conditions by which the compound (IV) is obtained from compound (I). Said conditions include an appropriate ratio between the polypeptide having beta-ketoacyl-CoA reductase activity and the substrate (i.e. the compound of formula I) in terms of concentration and appropriate conditions of salts, cofactors, pH and temperature as well to an appropriate reaction time.

Suitable salt conditions for carrying the third process according to the invention include, without limitation, any buffer commonly known such as phosphate buffer, a bicarbonate buffer, a carbonate buffer, Tris buffer, a borate buffer, a succinate buffer, a histidine buffer, a citrate buffer or a 2-(N-morpholino)ethanesulphonic acid buffer (MES) at concentrations of at least 10 mM, at least 20 mM, at least 30 mM, at least 40 mM, at least 50 mM, at least 60 mM, at least 70 mM, at least 80 mM, at least 90 mM, at least 100 mM, at least 200 mM, at least 300 mM or more. In a preferred embodiment, the third process according to the present invention is carried out in 100 mM MES buffer.

Suitable pH values for carrying the third process according to the invention include, without limitation, values of 4.5, 5, 5.5, 6, 6.5, 7, 7.5, 8, 8.5, 9 and 9.5. In a preferred embodiment, the process is carried out at a pH of about 6.

Suitable temperature conditions for carrying the third process according to the invention include, without limitation, about 20° C., about 25° C., about 30° C., about 35° C., about 37° C. In a preferred embodiment, the third process of the invention is carried out at 37° C.

Suitable reaction times for carrying the third process according to the invention include, without limitation, at least 1 ins, at least 1 s, at least 15 s, at least 30 s, at least 1 min, at least 2 min, at least 3 min, at least 4 min, at least 5 min, at least 6 min, at least 7 min, at least 8 min, at least 9 min, at least 10 min, at least 15 min, at least 20 min, at least 25 min, at least 30 min, at least 35 min, at least 40 min, at least 45 min, at least 50 min, at least 55 min, at least 1 h, at least 2 h, at least 3 h, at least 4 h, at least 5 h, at least 6 h, at least 7 h, at least 8 h, at least 9 h, at least 10 h or more. In a preferred embodiment, the reaction time is 1 hour.

Suitable concentration of the compound (I) for carrying the third process according to the invention include, without limitation, about 1 nM, about 5 nM, about 10 nM, about 20 nM, about 30 nM, about 40 nM, about 50 nM, about 100 nM, about 500 nM, about 1 μM, about 10 μM, about 20 μM, about 30 μM, about 40 μM, about 50 μM, about 100 μM, about 500 μM, about 1 mM, about 5 mM, about 10 mM, about 20 mM, about 30 mM, about 40 mM, about 50 mM, about 100 mM or more. In a preferred embodiment, the concentration of the compound of formula (I) is between 0.1 and 0.5 mM.

Suitable concentrations of the polypeptide having beta-ketoacyl-CoA reductase activity include, without limitation, about 1 μg/mL, about 2 μg/mL, about 3 μg/mL, about 5 μg/mL, about 8 μg/mL, about 10 μg/mL, about 20 μg/mL, about 30 μg/mL, about 50 μg/mL, about 75 μg/mL, about 100 μg/mL, about 200 μg/mL, about 300 μg/mL, about 500 μg/mL, about 750 μg/mL, about 1 mg/mL, about 2 mg/mL, about 3 mg/mL, about 5 mg/mL, about 7.5 mg/mL, about 10 mg/mL, about 20 mg/mL, about 30 mg/mL, about 50 mg/mL, about 75 mg/mL, about 100 mg/mL. In a preferred embodiment, the concentration of the polypeptide having beta-ketoacyl-CoA reductase activity is between 0.05 and 1 mg/mL.

The term “cofactor” as used herein refers to a non-protein chemical compound that is required for the protein's biological activity. In one embodiment, the molecule NADPH acts as cofactor in the process for obtaining a compound of formula (IV) defined above. In another embodiment, the molecule NADH acts as cofactor in the process for obtaining compound of formula (IV) defined above. NADH acts as a reducing agent that donates electrons which are necessary for the formation of the hydroxyl group (—OH) from the carbonyl group (═O). Consequently, NADH is transformed to its oxidized form, NAD⁺. Suitable concentrations of the NADPH or NADH cofactor for carrying the first process according to the invention include, without limitation, about 10 nM, about 50 nM, about 0.1 mM, about 0.5 mM, about 1 mM, about 5 mM, about 10 mM or more. In a preferred embodiment, the cofactor concentration is 1 mM.

In a particular and preferred embodiment of the third process of the invention, R₁ is an ethyl group. In another particular and preferred embodiment of the third process of the invention, R₂ is a methyl group.

Suitable polypeptides having beta-ketoacyl-CoA reductase activity for use according to the present invention have been detailed in the context of the first process of the invention and may be equally used in the context of the third process of the invention. In a particular and preferred embodiment of the third process of the invention, the polypeptide having beta-ketoacyl-CoA reductase activity consist of the amino acid sequence of the beta-ketoacyl-CoA reductase protein from Pseudomonas putida and which is shown in SEQ ID NO: 6.

### EXAMPLES

### Example 1. Alignment of Thiolase Sequences and Identification of Conserved Residues

The thiolases analyzed in the present study are shown in Table 1

TABLE 1
List of selected thiolases

| SEQ ID | | | | | | Identity* |
|---|---|---|---|---|---|---|
| NO | Enzyme | Organism | Length | Sequence ID | Plasmid | (%) |
| 2 | BktB | Ralstonia eutropha H16 | 394 | WP_011615089.1 | pET28mut | 100 |
| 1 | Erg10 | Saccharomyces cerevisiae s288 | 398 | NP_015297.1 | pET28mut | 39 |
| 3 | AtoB | Escherichia coli K-12 MG1655 | 394 | WP_000786547.1 | pET32b | 50 |
| 4 | TtThl | Thermus thermophilus HB8 | 401 | WP_011228352.1 | pET28mut | 45 |

*Referred to BktB sequence.

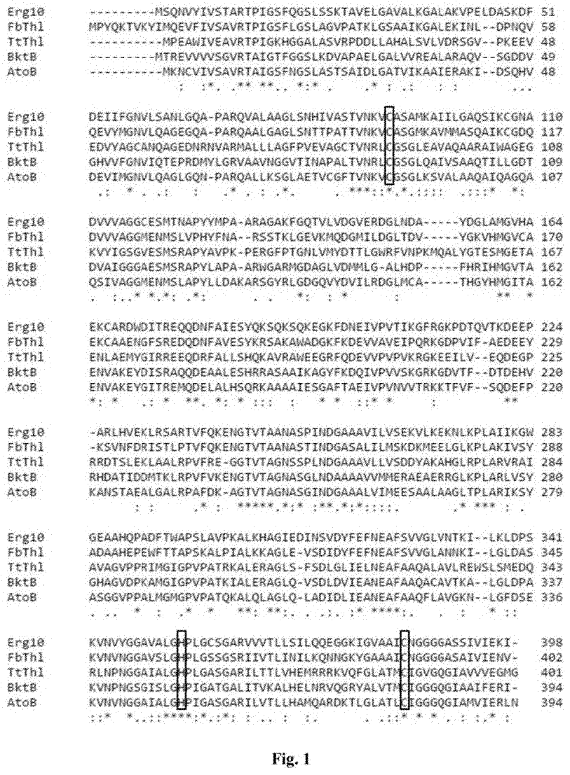

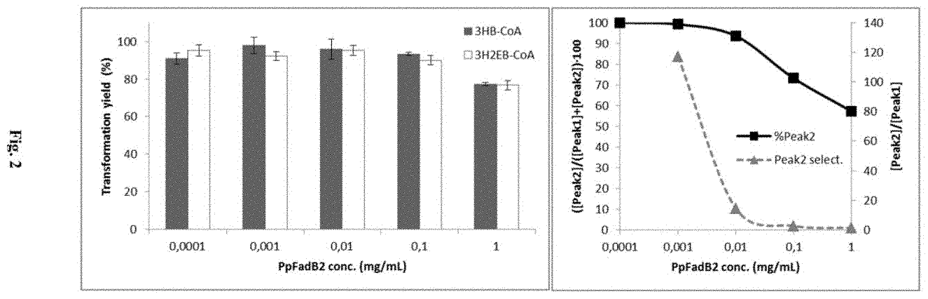

In order to indicate the degree of conservation of the selected residues, a multiple alignment was performed between the sequences of interest (SEQ ID NO: 1 to SEQ ID NO: 4). The alignment was performed using Clustal Omega web server from the European Bioinformatics Institute (EBI) using default parameters. The alignment is shown in FIG. 1.

### Example 2. Expression and Purification of Thiolases

Plasmids encoding natural or mutant thiolases were transformed into E. coli BL21 (DE3)-T1^{R}, expressed and purified as described below. The purified recombinant enzymes were then used as biocatalyst on the synthesis of 3-hydroxy-2-ethyl-butyryl-CoA (3H2EB-CoA).

Overnight culture in LB medium at 37° C. was inoculated to an OD_{600 nm} of 0.1 into 50 ml of NZY Auto-Induction LB medium (NZYTech, Portugal). NZY medium was supplemented with 100 μg/mL Kan or Amp (according to the corresponding plasmid—Table 1). The cultures were grown at 37° C. for 7 h and at 25° C. until 24 h aerobically. Hereafter the cells were collected by centrifugation at 4500 g for 10 minutes and kept at −20° C. until further purification.

Pellets (50 ml) were homogenized with 50 mM phosphate pH 7.5 (10 ml) and placed in an ice bath for sonication. Sonication took place for 10 minutes using pulse mode (5 seconds ON/OFF) and 20% amplitude. Lysates were centrifuged at 4° C., 10,000 g for 30 minutes. Supernatants containing the fusion protein were purified by His-affinity chromatography using empty PD-10 columns. Supernatants were incubated with 1 ml Talon® resin for 30 minutes at 4° C. Proteins were eluted with 1 ml elution buffer (300 mM imidazole, 50 mM phosphate pH 7.5), quantified and used for enzyme reaction.

### Example 3. High Throughput Expression and Purification of Thiolases

Plasmids encoding natural or mutant thiolases were transformed into E. coli BL21 (DE3)-T1^{R}, expressed and purified as described below. The purified recombinant enzymes were then used as biocatalyst on the synthesis of 3H2EB-CoA.

Overnight culture in LB medium at 37° C. was inoculated to an OD₆₀₀ nm of 0.1 in 5 ml of NZY Auto-Induction LB medium (NZYTech, Portugal). NZY medium was supplemented with 100 μg/mL kanamycin or ampicillin (according to the corresponding plasmid—Table 1). The cultures were aerobically grown at 37° C. for 7 h and at 25° C. until 24 h. Hereafter, cells were aliquoted in 96-deep-well plates and centrifuged at 4500 g for 10 minutes. Pellets were kept at −20° C. until further purification.

Pellets in 96-deep-well plates were resuspended with 250 μl lysis buffer (50 mM phosphate buffer pH 7.5, Bugbuster®, 10 μg/ml DNase I, 20 mM MgSO₄) for cell lysis. Cell lysis took place for 20 minutes. Lysates were centrifuged at room temperature, 10,000 g for 30 minutes. Supernatants containing the fusion protein were IMAC-purified. Supernatants were incubated with 100 μl Talon® resin for 15 minutes. Proteins were eluted with 50 μl elution buffer (300 mM imidazole, 50 mM phosphate buffer pH 7.5), quantified and used for enzyme reaction.

### Example 4. Assay for the Acyl-CoA Condensation by Thiolases

### a) Acetyl-CoA/Butyryl-CoA Condensation Assay

The acetyl-CoA/butyryl-CoA condensation activity of thiolase variants was evaluated in the synthetic direction by an in vitro coupled enzymatic assay with product profiling by HPLC-MS. Since the condensation reaction catalyzed by thiolases is endergonic, it was coupled with 3-hydroxy-2-methylbutyryl-CoA dehydrogenase from Pseudomonas putida (SEQ ID NO:6, PpFadB2) in order to drive the reaction in the forward direction (Thl/PpFadB2 coupled assay). Three reaction products were possible: 3-hydroxybutyryl-CoA (3HB-CoA) (wild type product of the condensation of 2 acetyl-CoA molecules), 3-hydroxyhexanoyl-CoA (3HH-CoA) (acetyl-CoA/butyryl-CoA linear condensation product) and 3-hydroxy-2-ethylbutyryl-CoA (3H2EB-CoA) (acetyl-CoA/butyryl-CoA branched condensation product). All three products were analyzed by HPLC-MS (see below).

Reactions were carried out in 2-(N-morpholino)ethanesulfonate (MES) buffer (pH 6.0), 100 μM acetyl-CoA, 100 μM butyryl-CoA, 1 mM NADH, 0.15 mg/ml PpFadB2 (SEQ ID NO:6) and 0.19 mg/ml of thiolase variant. Reaction mixtures were prepared in 96 well microplates in a 200 μL volume, and were incubated for 1 h at 37° C. with agitation (300 rpm in Thermomixer). Once completed, the enzyme was eliminated by filtration through 10 kDa cut-off filtration plates (Millipore). Samples were stored at −20° C. until further analysis.

### b) Acetyl-CoA/Acetyl-CoA Condensation Assay

Reactions were carried out in 2-(N-morpholino)ethanesulfonate (MES) buffer (pH 6) with 200 μM acetyl-CoA, 1 mM NADH, 0.15 mg/ml PpFadB2 and 0.19 mg/ml of thiolase variant. Reactions were processed as described in the previous section.

### Example 5. Expression and Purification of 3-Hydroxy-2-Methylbutyryl-CoA-Dehydrogenase from Pseudomonas putida (SEQ ID NO:6, PpFadB2)

Overnight culture in LB medium at 37° C. was inoculated to an OD_{600 nm} of 0.1 into 500 ml of NZY Auto-Induction LB medium (NZYTech, Portugal). NZY medium was supplemented with 100 μg/mL ampicillin. The cultures were grown at 37° C. for 7 h and at 25° C. until 24 h aerobically. Hereafter the cells were aliquoted into 50 ml and collected by centrifugation at 4500 g for 10 minutes and kept at −20° C. until further purification.

Pellets (50 ml) were homogenized with 50 mM phosphate pH 7.5 (20 ml) and placed in ice bath for sonication. Sonication took place for 10 minutes using pulse mode (5 seconds ON/OFF) and 20% amplitude. Lysates were centrifuged at 4° C., 10,000 g for 30 minutes. Supernatants containing the fusion protein were purified by His-Affinity chromatography using empty PD-10 columns. Supernatants were incubated with 3 ml Talon® resin for 30 minutes at 4° C. Proteins were eluted with 3 ml elution buffer (300 mM imidazole, 50 mM phosphate pH 7.5). Eluted solution was dialysed and concentrated by 10,000 MWCO ultrafiltration membrane (Amicon) to a final volume of approx. 0.5 ml. Purified protein was then quantified and kept at 4° C. until being used for enzyme reaction.

### Example 6. Characterization of the Dehydrogenase Activity of PpFadB2 Protein on 3-Keto-2-Ethylbutyryl-CoA

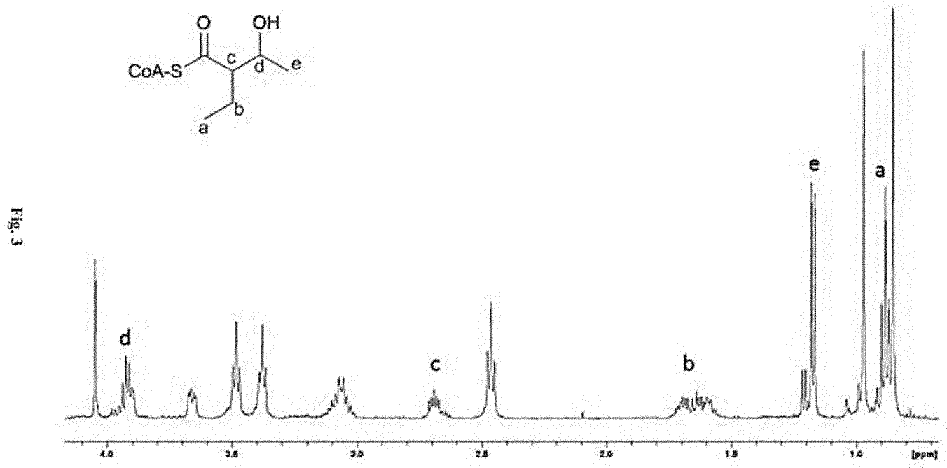

The FadB2 protein from Pseudomonas putida (PpFadB2; Accession Number: WP 064492895.1) exhibits a high activity on 3-keto-2-ethylbutyryl-CoA. To further confirm the biocatalytic potential of the enzyme, in vitro transformation assays were carried out using 1 mM NADH and 100 μM 3-keto-2-ethylbutyryl-CoA. Purified PpFadB2 was added to the assays to a final concentration of 0.0001-1 mg/mL. At all protein concentrations, the consumption of 3-keto-2-ethylbutyryl-CoA was total, and product yields were in the range of 76-95% as assessed by HPLC (FIG. 2).

The reduction of the keto group in 3-keto-2-ethylbutyryl-CoA generates a new chiral carbon in the molecule. Dehydrogenases are highly stereoselective, and preferential synthesis of one isomer is expected.

### LC-MS, LC-MS/MS and NMR Analysis of 3-Hydroxy-2-Ethyl-Butyryl-CoA and 3-Hydroxyhexanoyl-CoA

3-hydroxy-2-ethylbutyryl-CoA and 3-hydroxyhexanoyl-CoA were purchased from Sigma-Aldrich and analyzed by LC-MS. Chemically synthesized 3-hydroxy-2-ethylbutyryl-CoA eluted as two peaks with m/z ratio of 882.2. Chemically synthesized 3-hydroxyhexanoyl-CoA eluted as a single peak with m/z ratio of 882.2. All three peaks were well resolved chromatographically.

The MS/MS spectra of the two peaks from 3-hydroxy-2-ethylbutyryl-CoA were indistinguishable, and differed significantly from the MS/MS spectrum of the single peak of 3-hydroxy-2-ethylbutyryl-CoA (Table 2).

TABLE 2
MS/MS spectra of 3-hydroxy-2-ethylbutyryl-CoA (eluting as Peak 1 and Peak 2) and 3-hydroxyhexanoyl-CoA
(eluting as a single peak). MS/MS spectra were recorded in the negative mode.

| 3-hydroxy-2-ethylbutyryl-CoA | | | | | | | | |
|---|---|---|---|---|---|---|---|---|
| Peak 1 | | | Peak 2 | | | 3-hydroxyhexanoyl-CoA | | |
| m/z | Abund¹ | Norm² | m/z | Abund¹ | Norm² | m/z | Abund¹ | Norm² |
| 158.93 | 234289.31 | 2 | 158.93 | 97214.29 | 2 | 158.93 | 299574.63 | 5 |
| 408.01 | 1864911.63 | 16 | 408.01 | 750274.81 | 15 | 408.01 | 2362461.75 | 17 |
| 409.02 | 223195.11 | 2 | 409.01 | 84708.17 | 2 | | | |
| 426.02 | 1443406.88 | 12 | 426.02 | 570374.19 | 12 | 426.02 | 1848262.50 | 13 |
| | | | | | | 453.15 | 292756.75 | 2 |
| 533.11 | 823315.06 | 7 | 533.11 | 317675.25 | 6 | 533.11 | 934316.13 | 7 |
| 551.12 | 287170.13 | 2 | 551.12 | 93560.85 | 2 | | | |
| | | | | | | 782.20 | 670456.69 | 5 |
| 800.21 | 549242.69 | 5 | 800.21 | 199489.63 | 4 | 800.21 | 668001.81 | 5 |
| 880.18 | 3875833.50 | 33 | 880.18 | 1817105.25 | 37 | 880.18 | 4211549.50 | 30 |
| 881.18 | 1724722.75 | 15 | 881.18 | 717834.38 | 15 | 881.18 | 1956559.88 | 14 |
| 882.18 | 669350.13 | 6 | 882.18 | 267766.75 | 5 | 882.18 | 755951.38 | 5 |

¹Abund: absolute value of peak area integration.
²Norm: relative value of peak area integration, normalized by the sum of all peaks integration areas in MS/MS spectrum.

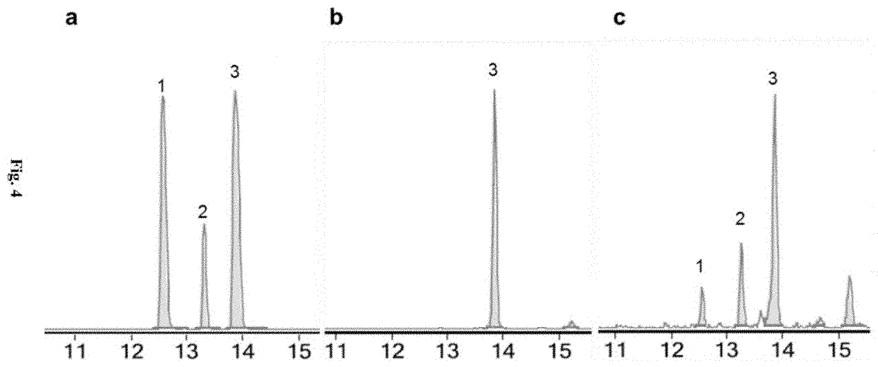

Chemically synthesized 3-hydroxy-2-ethylbutyryl-CoA was further analyzed by ¹H-NMR, which further confirmed the presence of two isomers (FIG. 3). Both LC-MS and ¹H-NMR confirmed that Peak 1 and Peak 2 isomers were present in a 7:3 ratio in chemically synthesized 3-hydroxy-2-ethylbutyryl-CoA.

Thus, it was concluded that chemically synthesized 3-hydroxy-2-ethylbutyryl-CoA consisted of a mixture of two diastereoisomers in a 7:3 ratio.

### Experimental Assessment of the Stereoselectivity of PpFadB2.

In the in vitro assays, the stereoselectivity of the enzyme was highly affected by its concentration. At 1 mg/mL PpFadB2, reduction of 3-keto-2-ethyl-butyryl-CoA to 3-hydroxy-2-ethylbutyryl-CoA yields a mixture of two diastereoisomers which are resolved by HPLC. At lower enzyme concentrations, the ratio of isomers changed, with increasing yield in the Peak 2 isomer (FIG. 2).

### Example 7. Acetyl-CoA/Butyryl-CoA Condensation Activity of Thiolase Variants

BktB_L89I_M290A_M379L (Var10) variant was constructed, expressed and purified to be used as biocatalyst in the synthesis of 3H2EB-CoA.

This protein variant was able to catalyze the synthesis of the desired compound (see chromatogram in Figure. 4).

Once Var10 was identified as a biocatalyst able to perform the synthesis of 3H2EB-CoA, we decided to investigate the effect of those mutations on the other studied thiolases (Table 3). The selected thiolases show sequence identities lower than 50% (Table 3).

TABLE 3
List of thiolase variants homologue to BktB Var10 mutant.

| Thiolase | Name |
|---|---|
| BktB_L89I_M290A_M379V | BktB_Var10 |
| AtoB_V87I_M289_L378V | AtoB_Var10 |
| Erg10_V90I_F293A_I383V | Erg10_Var10 |
| TtThl_L88I_M294A_M385V | TtThl_Var10 |

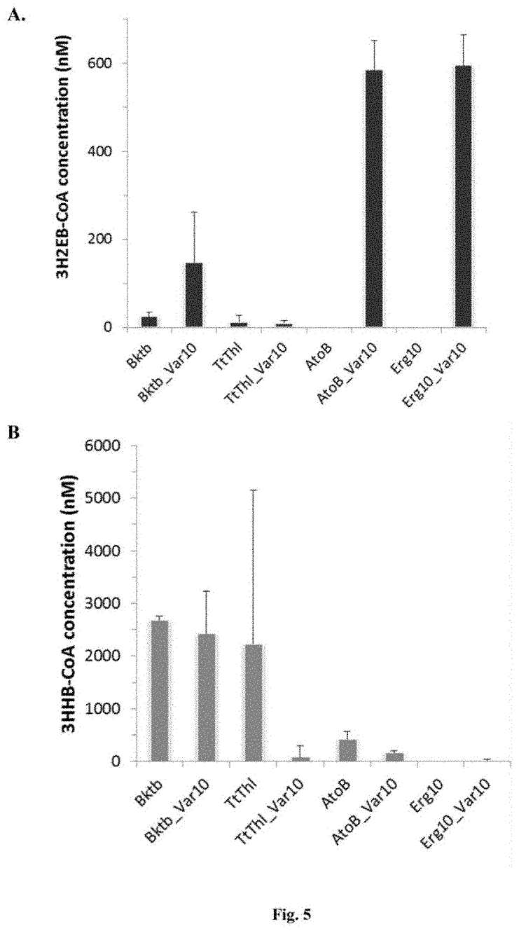

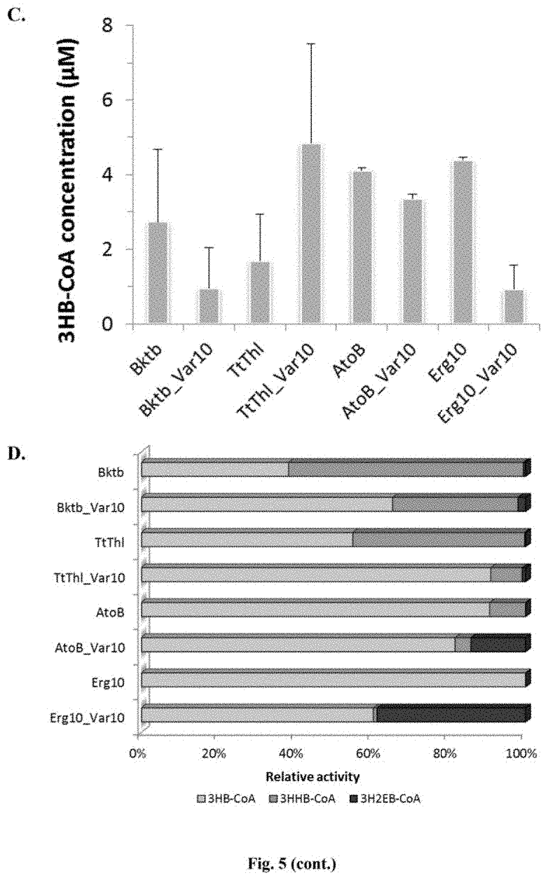

The four modified thiolases were constructed, purified and used as biocatalysts for the in vitro acetyl-CoA/butyryl-CoA condensation. FIG. 5 displays yield of products A) 3H2EB-CoA, B) 3HHB-CoA, C) 3HB-CoA and D) product selectivity for wild type thiolases and their triple mutants.

Regarding acetyl-CoA condensation activity, BktB_Var10, AtoB_Var10 and Erg10_Var10 showed worse activity than their wild type enzymes for 3HB-CoA synthesis (FIG. 5).

Remarkably AtoB_Var10 and Erg10_Var10 improved acetyl-CoA/butyryl-CoA branched condensation product yields, exhibiting comparable activity (ca. 0.6% product yield), 4-fold higher than that of BtkB Var10. Also, Erg10_Var10 showed a remarkable selectivity towards the synthesis of the branched compound (3HB-CoA/3H2EB-CoA 60:40).

In the case of the synthesis of 3HH-CoA, BktB and its Var10 were the best performers, having a comparable synthetic activity. Also AtoB_Var10 was able to catalyse that reaction, although giving rise to lower yields. Mutations on TtThl killed the linear condensation activity. Interestingly, Erg10_Var10 produced traces of the linear product that were close to the detection limits (activity not observed in the wild type enzyme). Comparing, Erg10_Var10 and AtoB_Var10 showed 43 and 3 times higher yields for the branched compound than for the linear one, respectively.

Therefore, Erg10_Var10 was the best variant since it accepts substrates other than acetyl-CoA in the second step of the Claisen condensation reaction, while being selective towards the synthesis of branched compounds.

### Example 8. Analysis of Single and Double Mutants of Erg10 Thiolase

We generated all possible combinations of the variations at these three positions. The three single variants (Erg10_V90I, Erg10_F293A and Erg10_I383V) and the three double variants (Erg10_V90I_F293A, Erg10_V90I_I383V and Erg10_F293A_I383V) were evaluated for the synthesis of 3H2EB-CoA.

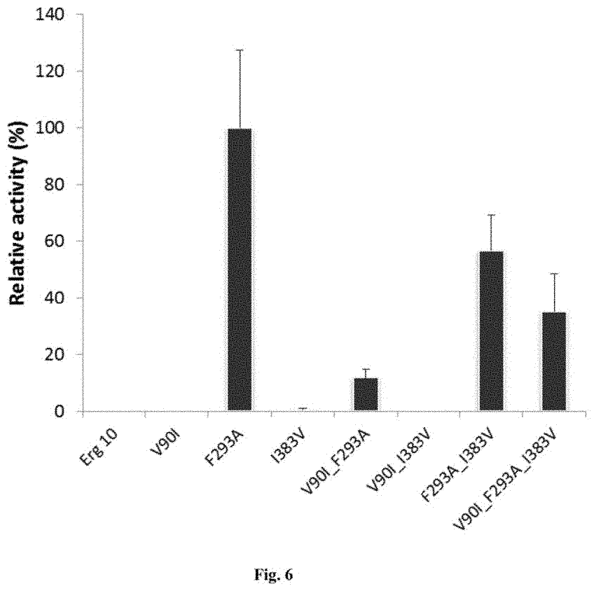

Double variants having the mutation F293A on their sequences were able to catalyse the condensation of acetyl-CoA and butyryl-CoA, giving rise to 3H2EB-CoA (FIG. 6). Of those having activity, F293A_I383V displayed higher performance than V90I_F293A.

Concerning single mutants, only F293A was capable of carrying out the desired reaction, showing 3-fold increase of activity compared to Erg10_Var10. In the case of single variant I383V, the branched compound synthesised was close to the detection limits. Acetyl-CoA/butyryl-CoA condensation products were not found for variant V90I (FIG. 6).

### Example 9. Analysis of Erg10 Thiolase Mutants at Phe293

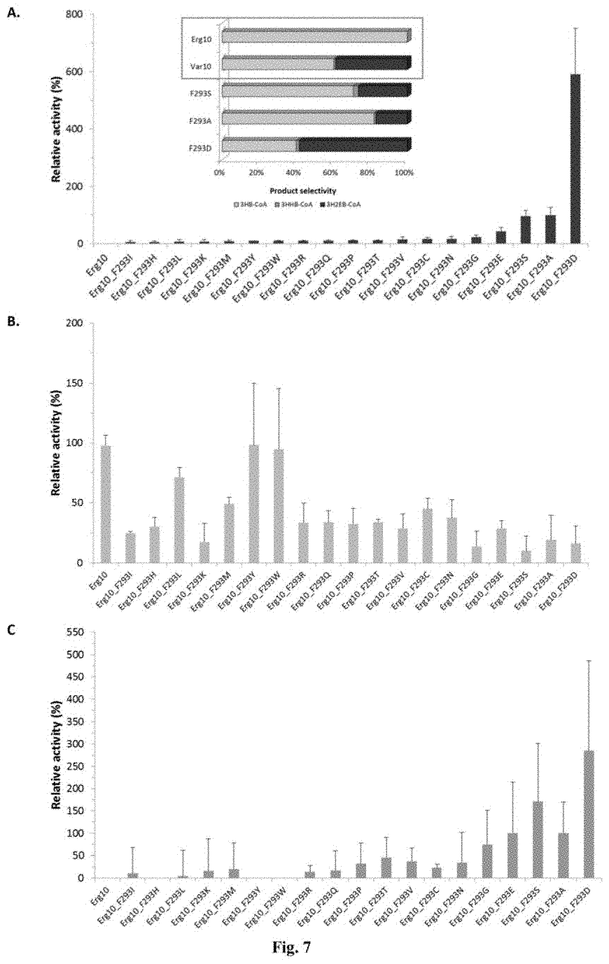

The role of Phe293 residue for thiolase activity of Erg10 was explored by saturation mutagenesis (changing it for the remaining 19 natural amino acids). Surprisingly, the F293D mutant outperformed Erg10_Var10 17 times and 120 times the initial activity observed in BktB. (FIG. 7).

When looking at product selectivity (FIG. 7A insert), F293D came up as the most active and selective variant for the production of 3H2EB-CoA. Remarkably, this mutant yielded 1.5- and 35-times more 3H2EB-CoA than 31-1B-CoA (FIG. 7B) and 3HH-CoA. (FIG. 7C), respectively.

### Example 10. Kinetic Analysis of Thiolase Variants

For the kinetic analysis of the acetyl-CoA/butyryl-CoA condensation activity of the best thiolase variants, the concentration of enzyme in the assay was adjusted in order to ensure capturing the linear phase of product synthesis profile. Two different enzyme concentrations were assayed for each protein (0.04 and 0.02 mg/ml for Erg10_F293D; 0.19 and 0.09 mg/ml for Erg10_F293A). Reactions were followed during 24 h. Specific activity was calculated for the first 20 minutes of reaction.

Assays were carried out in triplicate and at least at two protein concentrations in order to ensure the reliability of the activity determined.

Reactions were stopped by fast separation of the enzyme by filtration through 10 kDa cut-off filtration plates. Samples were stored at −20° C. before until further analysis.

Specific activity for Erg10_F293D, was measured and compared to the one for Erg10_F293A. Results showed Erg_F293D was 12 times more active for the synthesis of 3H2EB-CoA than Erg_F293A, displaying a specific activity of 700±140 mU/g protein and 54±10 mU/g protein, respectively.

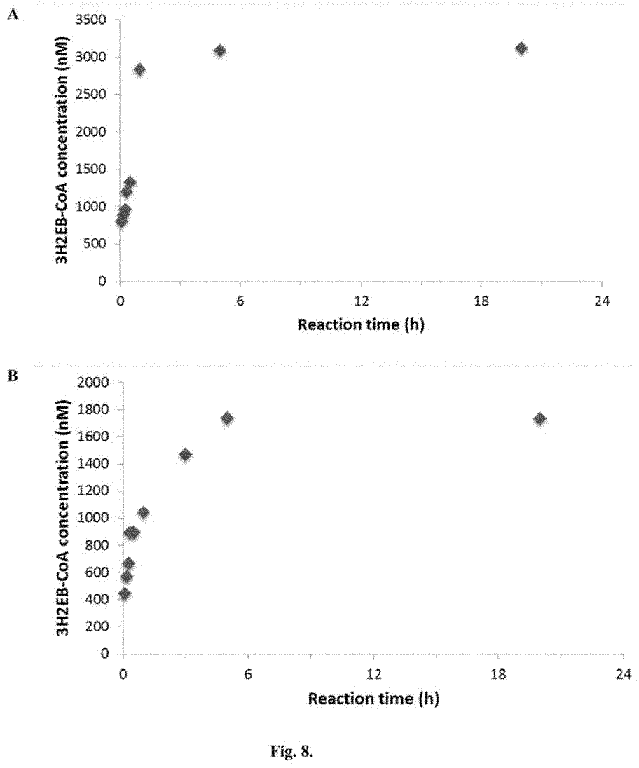

We carried out a time course of 3H2EB-CoA formation. Both enzymes showed a typical saturation curve (FIG. 8). After 24 h, we observed a maximum production yields for Erg10_F293D and Erg10_F293A ca. 3000 nM and 1800 nM, respectively (FIG. 8).

### Example 11: Characterization of Substrate Flexibility of Erg10 Thiolase Variants

We have analyzed the capability of Erg10 variants to catalyze several condensation reactions in vitro. The enzyme assays were carried out as described for the acetyl-CoA/butyryl-CoA condensation reaction but with alternative substrates.

We performed Cross-Claisen condensation reactions using acetyl-CoA/propionyl-CoA and acetyl-CoA/hexanoyl-CoA as substrate pairs. Reactions were analyzed by LC-MS by monitoring chromatograms for compounds with m/z of 868.1 or 910.2, respectively.

### Acetyl-CoA/Propionyl-CoA Cross Claisen Condensation.

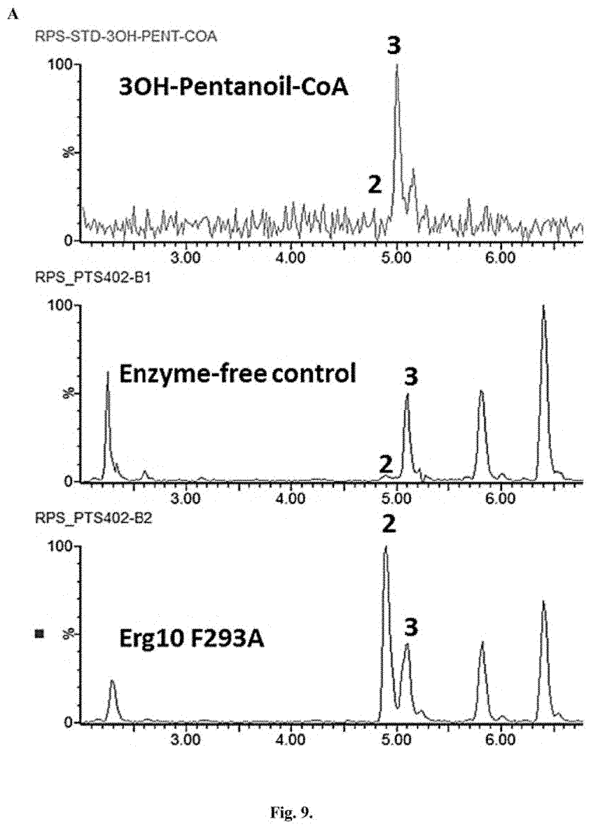

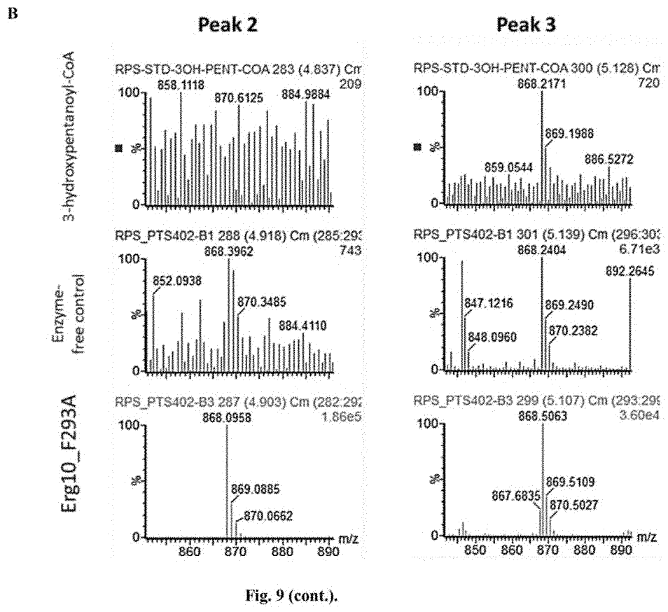

Chromatograms were monitored for compounds with m/z of 868.1. Several peaks were detected in samples, two of them (peaks 2 and 3) were significantly more intense (13 and 1.5-fold higher in reaction samples with Erg10_F293A compared to enzyme-free controls; 122 and 5-fold higher in reaction samples with Erg10_F293D compared to enzyme-free controls) (FIG. 9). These two peaks therefore correspond to products generated by the action of Erg10 variants on substrates.

As in the case of the acetyl-CoA/butyryl-CoA condensation, linear (3-hydroxypentanoyl-CoA) and branched (3-hydroxy-2-methylbutyryl-CoA) condensation products are possible. The retention time of peak 2 differs to that of the commercial standard 3-hydroxypentanoyl-CoA (Sigma), which suggests that the major product generated in the reaction is likely to correspond to the branched condensation product (3-hydroxy-2-methylbutyryl-CoA, peak 2), although the linear condensation product is also formed (3-hydroxypentanoyl-CoA, peak 3) (FIG. 9).

### Acetyl-CoA/Hexanoyl-CoA Cross Claisen Condensation.

Chromatograms were monitored for compounds with m/z of 910.2. Several peaks were detected in samples, one of them (retention time 12.5 min) was significantly more intense in reaction samples compared to enzyme-free controls (22-fold higher in reaction samples with Erg10_F293D compared to enzyme-free controls). This peak therefore corresponds to a product generated by the action of Erg10 variant on substrates (FIG. 10).

Linear (3-hydroxyoctanoyl-CoA) and branched (3-hydroxy-2-butylbutyryl-CoA) condensation products are possible but its identity could not be confirmed, due to the lack of a commercial standard.

Thus, the results from acetyl-CoA/propionyl-CoA and acetyl-CoA/hexanoyl-CoA Cross-Claisen condensation reactions suggest that:
Erg10_F293D variant can accept a wide range of acyl-CoAs as substrate in the second step of the Claisen condensation.
Erg10_F293D variant is highly specific towards acetyl-CoA as substrate in the first step of the Claisen condensation.
Altogether, these two observations further support the higher selectivity of Erg10_F293 variants towards the synthesis of branched condensation compounds.

### Example 12. Acetyl-CoA/Acetyl-CoA Condensation Activity of Erg10 Thiolase Variants

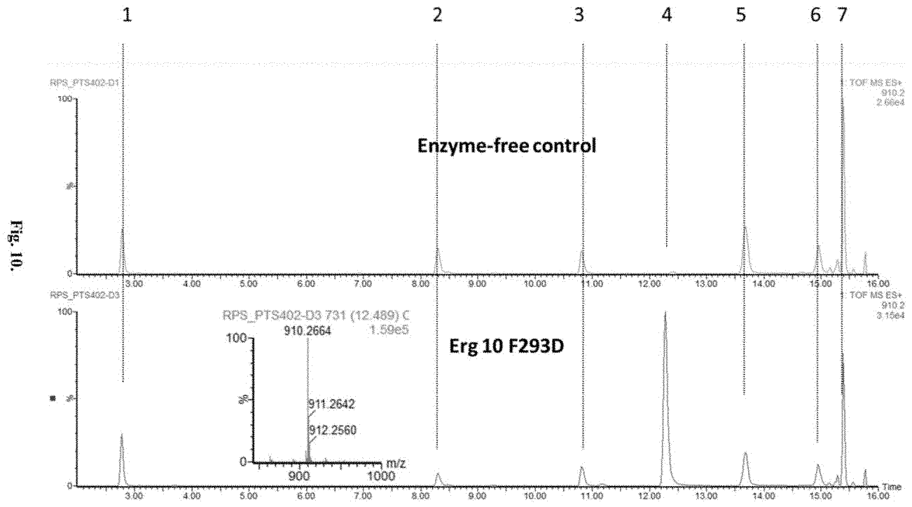

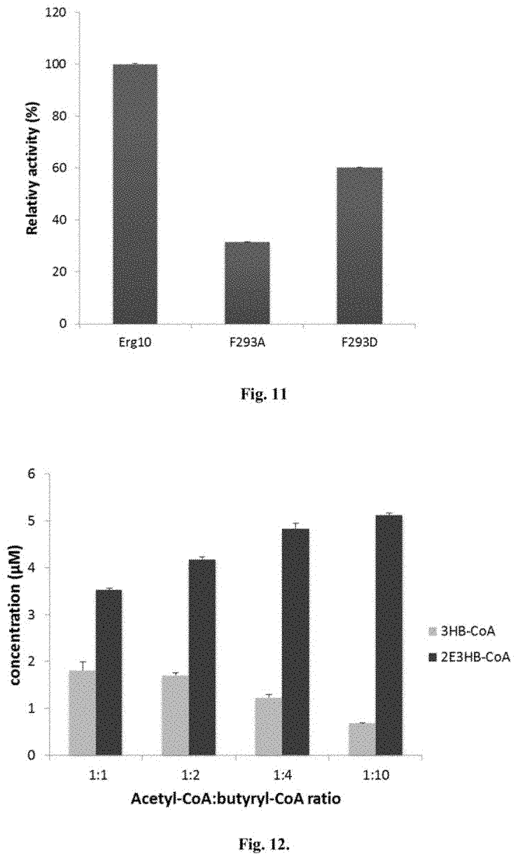

Wild type reaction was also carried out with these enzymes. Erg10_F293D and Erg10_F293A retained 60% and 30%, respectively, of the acetyl-CoA/acetyl-CoA condensation activity of the wild type enzyme (FIG. 11).

### Example 13: Effect of Acetyl-CoA/Butyryl-CoA Ratio on the Product Formation Profile in Acetyl-CoA/Butyryl-CoA Condensation Assays

Different ratios acetyl-CoA:butyryl-CoA were assayed. We evaluated acetyl-CoA:butyryl-CoA ratios from 1:1 to 1:10. An increase of 3-hydroxy-2-ethylbutyryl-CoA product formation was observed when increasing butyryl-CoA concentration. Contrarily, 3HB-CoA production was significantly reduced under the same reaction conditions (FIG. 12). Regarding 3HH-CoA, a clear tendency was not observed due to the low concentration near to detection limits in all cases.

### Example 14: Analysis of Single and Double Mutants of BktB Thiolase (SEQ. ID NO. 2)

Ralstonia eutropha thiolase (BktB, SEQ. ID. NO:2) is able to condense two acetyl-CoA molecules (yielding acetoacetyl-CoA) or one acetyl-CoA and one butyryl-CoA molecules (yielding 3-ketohexanoyl-CoA). A small amount of 3-keto-2-ethylbutyryl-CoA is produced. In the presence of PpFadB2 these compounds are transformed to 3-hydroxybutyryl-CoA, 3-hydroxy-2-ethylbutyryl-CoA and 3-hydroxyhexanoyl-CoA, respectively.

Single, double and triple mutants at Leu89, Met290 and Met379 were constructed. Seven protein variants were constructed: BktB Leu89Ile, BktB Met290Ala, BktB Met290Asp, BktB Met379Val, BktB Leu89Ile Met290Ala, BktB Leu89Ile Met379Val, BktB Met290Ala Met379Val or BktB Leu89Ile Met290Ala Met379Val. The ability to conduct a Claisen condensation activity on a mixture of acetyl-CoA and butyryl-CoA of the resulting protein variants was assessed as explained before.

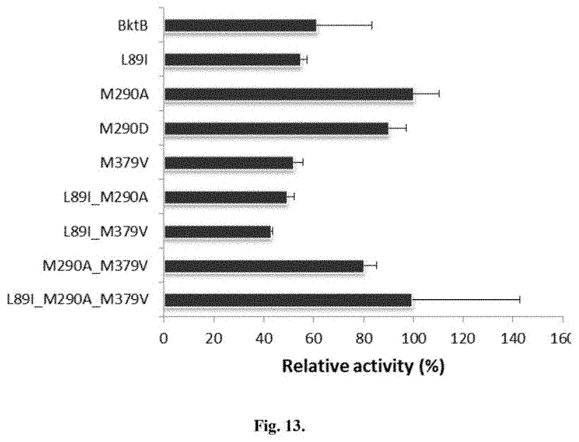

Reaction products identified were: 3-hydroxybutyryl-CoA, 3-hydroxy-2-ethylbutyryl-CoA and 3-hydroxyhexanoyl-CoA. Two different isomers of 3-hydroxy-2-ethylbutyryl-CoA were detected, which eluted at different retention times (FIG. 13).

### Example 15: Analysis of Single and Triple Mutants of AtoB Thiolase (SEQ. ID NO. 3)

Escherichia coli thiolase (AtoB, SEQ. ID. NO:3) is able to condense two acetyl-CoA molecules (yielding acetoacetyl-CoA) or one acetyl-CoA and one butyryl-CoA molecules (yielding 3-ketohexanoyl-CoA). In the presence of PpFadB2 these compounds are transformed to 3-hydroxybutyryl-CoA and 3-hydroxyhexanoyl-CoA, respectively.

Single and triple mutants at Val87, Met289 and Leu378 were constructed. Three protein variants were constructed: AtoB Met289Ala, AtoB Met289Asp and AtoB Val87Ile Met289Ala Leu378Val (Var10). The ability to conduct a Claisen condensation activity on a mixture of acetyl-CoA and butyryl-CoA of the resulting protein variants was assessed as explained before.

Reaction products identified were: 3-hydroxybutyryl-CoA, 3-hydroxy-2-ethylbutyryl-CoA and 3-hydroxyhexanoyl-CoA. Two different isomers of 3-hydroxy-2-ethylbutyryl-CoA were detected, which eluted at different retention times (Table 4).

TABLE 4
Concentrations of the products of the acetyl-CoA/butyryl-CoA
condensation reaction (3-hydroxybutyryl-CoA, 3-hydroxy-2-
ethylbutyryl-CoA and 3-hydroxy-hexanoyl-CoA) catalyzed by
AtoB thiolase and its mutants. Reactions were carried out
with a 1:1 acetyl-CoA:butyryl-CoA ratio.

| | 3HB-CoA (μM) | 3H2EB-CoA (nM) | 3HHB-CoA (nM) |
|---|---|---|---|
| AtoB | 4.12 | 0 | 425 |
| M289A | 5.62 | 33.62 | 17.22 |
| M289D | 12.51 | 2491.38 | 34.57 |
| Var10 | 11.63 | 2166.47 | 30.92 |

### Example 16. UPLC-MS Analysis of Coenzyme A Thioesters

Standard stock solutions were prepared in dH₂O. Then, calibration curve samples containing different amounts of each analyte were obtained after dilution with 100 mM ammonium formate buffer. The final concentrations were 40, 30, 20, 10, 4, 1, 0.1 mM for acetyl-CoA, butyryl-CoA and 3-hydroxybutyryl-CoA and 200, 150, 100, 50, 20, 5, 2, 0.5 μM for 3-hydroxyhexanoyl-CoA and 3-hydroxy-2-ethylbutyryl-CoA. Blank samples were also included before and after the calibration curve samples in order to control the absence of carry-over effect.

Chromatography was performed in an Acquity UPLC system using an Acquity BEH C18 column (100×2.1 mm, 1.7 μm) from Waters (Milford, Mass., USA) and equipped with photodiode array detector (PDA). The gradient elution buffers were A (water and 100 mM of ammonium formate) and B (acetonitrile). The gradient method was as follows: 0-1 min at 95% A, 1-14 min to 80% A, 14-15 min to 10% A 15-17 min at 10% A, 17-17.5 min to 95% A, 17.5-20 min at 95% A. The UV detector wavelength was set at 254 nm and the injection volume was 10 μL. Total run time was 20 min and the flow rate was set at 300 μLmin-1.

The mass spectrometry detection was carried out using a time-of-flight mass spectrometer (ESI-TOF) LCT Premier XE from Waters (Milford, Mass., USA) with an electrospray ionization source, working in positive /V mode. The MS range acquired was between m/z 100-1000. The capillary and cone voltages were set at 3000 and 100 V, respectively. For other parameters, desolvation gas temperature was 220° C. and source temperature was 120° C. The desolvation gas flow was set at 600 L·h⁻¹ and cone gas flow was set at 50 L·h⁻¹.

Masslynx v4.1 software was used to analyze chromatograms and spectra (Waters, Milford, Mass., USA). All the analytes were identified by mass spectrometry (Table 5). The quantification of 3-hydroxyhexanoyl-CoA and 3-hydroxy-2-ethylbutyryl-CoA was performed by ESI-TOF MS. On the other hand, in order to avoid the signal saturation, the quantification of acetyl-CoA, butyryl-CoA and 3-hydroxybutyryl-CoA was performed by UV absorbance at 254 nm.

TABLE 5
Analytes, retention time, adduct and detected ion.

| Analyte | Retention time (min) | Adduct | m/z |
|---|---|---|---|
| Acetyl-CoA | 2.9-3.2 | [M + H]+ | 810.12 |
| 3-hydroxybutyryl-CoA | 3.2-3.5 | [M + H]+ | 854.16 |
| 3-hydroxy-2-ethylbutyryl- | 5.9-6.2 | [M + H]+ | 882.19 |
| CoA isomer 1 | | | |
| 3-hydroxy-2-ethylbutyryl- | 6.6-6.9 | [M + H]+ | 882.19 |
| CoA isomer 2 | | | |
| 3-hydroxyhexanoyl-CoA | 7.2-7.5 | [M + H]+ | 882.19 |
| Butyryl-CoA | 7.4-7.7 | [M + H]+ | 838.16 |

1. A mutant thiolase capable of catalyzing the formation of a compound of formula (I) 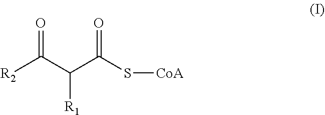
by the condensation of a compound of formula (II) 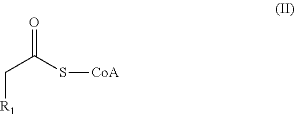
with a compound of formula (III) 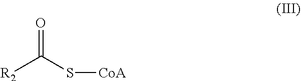
wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A,
said mutant thiolase having at least a mutation at a position corresponding to position 293 in the polypeptide of SEQ ID NO: 1 wherein the thiolase shows at least a 75% sequence identity with a thiolase selected from the group consisting of SEQ ID NO:1, 2, 3 or 4, with the proviso that said thiolase is not a thiolase consisting of the sequence SEQ ID NO: 7 or SEQ ID NO: 8.

2. The thiolase according to claim 1, wherein the sequence in said thiolase corresponding to the thiolase signature having the accession number PS00098 in the Prosite database has an identity of at least 35% to the sequence corresponding to said signature in the polypeptide of SEQ ID NO:1, wherein the sequence in said thiolase corresponding to the thiolase signature having the accession number PS00737 in the Prosite database has an identity of at least 50% to the sequence corresponding to said signature in the polypeptide of SEQ ID NO:1 and/or wherein the sequence in said thiolase corresponding to the thiolase signature having the accession number PS00099 in the Prosite database has an identity of at least 25% to the sequence corresponding to said signature in the polypeptide of SEQ ID NO:1.

3. The thiolase according to claim 2, wherein the mutant thiolase is selected from the group consisting of:
(i) The polypeptide of SEQ ID NO: 1, wherein the at least one mutation is located at position Phe293;
(ii) The polypeptide of SEQ ID NO: 2 wherein the at least one mutation is located at position Met290,
(iii) The polypeptide of SEQ ID NO: 3, wherein the at least one mutation is located at position Met289 and
(iv) The polypeptide of SEQ ID NO: 4, wherein the at least one mutation is located at position Met294.

4. The thiolase according to claim 3, wherein the mutant thiolase is the polypeptide of SEQ ID NO: 1 wherein the at least one mutation located at position Phe293 is selected from the group consisting of Phe293Asp, Phe293Ala, Phe293Ser, Phe293Gly and Phe293Glu.

5. The thiolase according to claim 3, wherein the mutant thiolase is the polypeptide of SEQ ID NO: 2 wherein the at least one mutation at position Met290 is selected from the group consisting of Met290Asp, Met290Ala, Met290Ser, Met290Gly and Met290Glu.

6. (canceled)

7. The thiolase according to claim 3, wherein the mutant thiolase is the polypeptide of SEQ ID NO: 3, wherein at least one mutation located at position Met289 is selected from the group consisting of Met289Asp, Met289Ala, Met289Ser, Met289Gly and Met289Glu.

8. (canceled)

9. The thiolase according to claim 3, wherein the mutant thiolase is the polypeptide of SEQ ID NO: 4 wherein the at least one mutation located at position Met294 is selected from the group consisting of Met294Asp, Met294Ala, Met294Ser, Met294Gly, and Met294Glu.

10-20. (canceled)

21. A process for the production of a compound of formula (I) comprising contacting a compound of formula (II) and a compound of formula (III) in the presence of a mutant thiolase capable of catalyzing the formation of a compound of formula (I) 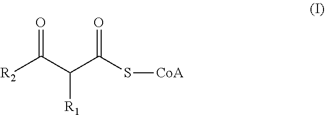
by the condensation of a compound of formula (II) 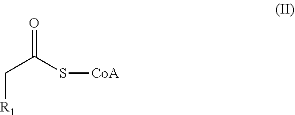
with a compound of formula (III) 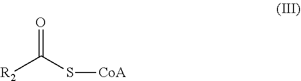
wherein R₁ and R₂ are independently selected from the group consisting of linear or branched C₁-C₆ alkyl, linear or branched C₂-C₆ alkenyl, linear or branched C₂-C₆ alkynyl, aryl, aryl-alkyl and benzyl, and S-CoA is coenzyme A,
said mutant thiolase having at least a mutation with respect to the naturally-occurring thiolase, wherein said mutation is located at a position corresponding to position 293 in the polypeptide of SEQ ID NO: 1; and wherein said contacting is carried out under conditions adequate for the condensation of compounds of formula (II) and (III) into a compound of formula (I).

22. The process according to claim 21 wherein the mutant thiolase shows at least a 75% sequence identity with a thiolase selected from the group consisting of SEQ ID NO:1, 2, 3 or 4.

23. The process according to claim 21 wherein the thiolase is not a thiolase consisting of the sequence SEQ ID NO: 7 or SEQ ID NO: 8.

24. The process according to claim 21, wherein the sequence in said thiolase corresponding to the thiolase signature having the accession number PS00098 in the Prosite database has an identity of at least 35% to the sequence corresponding to said signature in the thiolase of SEQ ID NO:1, wherein the sequence in said thiolase corresponding to the thiolase signature having the accession number PS00737 in the Prosite database has an identity of at least 50% to the sequence corresponding to said signature in the thiolase of SEQ ID NO:1 and/or wherein the sequence in said thiolase corresponding to the thiolase signature having the accession number PS00099 in the Prosite database has an identity of at least 25% to the sequence corresponding to said signature in the thiolase of SEQ ID NO:1.

25. The process according to claim 21, wherein the mutant thiolase is selected from the group consisting of:
(i) The polypeptide of SEQ ID NO: 1 wherein the at least one mutation is located at position Phe293;
(ii) The polypeptide of SEQ ID NO: 2 wherein the at least one mutation is located at position Met290;
(iii) The polypeptide of SEQ ID NO: 3, wherein the at least one Mutation is located at position Met289 and;
(iv) The polypeptide of SEQ ID NO: 4, wherein the at least one mutation is located at position Met294.

26. The process according to claim 25, wherein the mutant thiolase is the polypeptide of SEQ ID NO: 1 wherein the at least one mutation located at position Phe293 is selected from the group consisting of Phe293Asp, Phe293Ala, Phe293Ser, Phe293Gly and Phe293Glu.

27. The process according to claim 25, wherein the mutant thiolase is the polypeptide of SEQ ID NO: 2 wherein the at least one mutation at position Met290 is selected from the group consisting of Met290Asp, Met290Ala, Met290Ser, Met290Gly and Met290Glu.

28. The process according to claim 27, wherein the mutant thiolase is the polypeptide of SEQ ID NO: 2 wherein the at least one mutation at position Met290 is selected from the group consisting of Met290Asp, and Met290Ala.

29. The process according to claim 25, wherein the mutant thiolase is, the polypeptide of SEQ ID NO: 3, wherein at least one mutation located at position Met289 is selected from the group consisting of Met289Asp, Met289Ala, Met289Ser, Met289Gly and Met289Glu.

30. The process according to claim 29, wherein the mutant thiolase is the polypeptide of SEQ ID NO: 3, wherein at least one mutation located at position Met289 is selected from the group consisting of Met289Asp and Met289Ala.

31. The process according to claim 25, wherein the mutant thiolase is the polypeptide of SEQ ID NO: 4 wherein the at least one mutation located at position Met294 is selected from the group consisting of Met294Asp, Met294Ala, Met294Ser, Met294Gly, and Met294Glu.

32-42. (canceled)

43. Process for the production of a compound of formula (IV) comprising contacting a compound of formula (I) with a polypeptide having beta-ketoacyl-CoA reductase activity capable of catalyzing the formation of a compound of formula (IV) 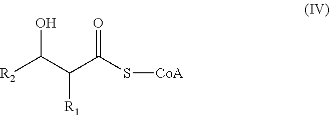
from the compound of formula (I), wherein R₁, R₂ and S-CoA are as defined in claim 1.

44. The process according to claim 43 wherein the polypeptide having beta-ketoacyl-CoA reductase activity consists of the sequence of SEQ ID NO: 6.

45-46. (canceled)
